(19) United States
(12) Patent Application Publication
(10) Pub. No.: US 2025/0388681 A2
(43) Pub. Date: Dec. 25, 2025

(54) Dosing Regimen for Therapies Comprising Bispecific Anti-EGFR/C-Met Antibodes

(71) Applicant: Janssen Biotech, Inc., Horsham, PA (US)

(72) Inventor: Roland Knoblauch, Philadelphia, PA (US)

(21) Appl. No.: 18/222,093

(22) Filed: Jul. 14, 2023

Related U.S. Application Data
(60) Provisional application No. 63/389,041, filed on Jul. 14, 2022.

(51) Int. Cl. C07K 16/28; A61K 31/282; A61K 31/519; A61K 31/5377; A61K 39/00; A61K 39/395; A61P 35/00

(52) U.S. Cl. CPC C07K 16/2863; A61K 31/282; A61K 31/519; A61K 31/5377; A61K 39/39558; A61P 35/00; A61K 2039/505; A61K 2039/545; C07K 2317/31; C07K 2317/41

(57) ABSTRACT

Provided are methods of treating an epidermal growth factor receptor (EGFR)-expressing or hepatocyte growth factor receptor (c-Met)-expressing cancer in a subject in need thereof. The methods comprise administering to the subject a therapy comprising an isolated bispecific anti-EGFR/c-Met antibody, wherein the administration comprises a dose of about 1400-2100 mg, administered once per a 21-day cycle.

### CROSS-REFERENCE TO RELATED APPLICATIONS

This application claims the benefit of U.S. Provisional Patent Application No. 63/389,041, filed Jul. 14, 2022, the disclosure of which is herein incorporated by reference in its entirety.

### REFERENCE TO SEQUENCE LISTING SUBMITTED ELECTRONICALLY

The sequence listing of the present application is submitted electronically via The United States Patent and Trademark Center Patent Center as an XML formatted sequence listing with a file name “JBI6724USNP1SEQLIST.xml”, creation date of Jul. 14, 2023, and a size of 21 kilobytes (KB). This sequence listing submitted is part of the specification and is herein incorporated by reference in its entirety.

### FIELD OF THE INVENTION

The present invention relates to methods of treating an epidermal growth factor receptor (EGFR)-expressing or hepatocyte growth factor receptor (c-Met)-expressing cancer in a subject in need thereof. The methods comprise administering to the subject a therapy comprising an isolated bispecific anti-EGFR/c-Met antibody, wherein the administration comprises a dose of about 1400-2100 mg, administered once per a 21-day cycle.

### BACKGROUND OF THE INVENTION

The roles of epidermal growth factor receptor (EGFR, ErbB1 or HER1) and hepatocyte growth factor receptor (c-Met) in cancer are well-established, making these attractive targets for combination therapy. Both receptors signal through the same ERK and AKT survival and anti-apoptotic pathways and often are upregulated as a resistant mechanism for single agent treatment. Additionally, relapse or resistance to existing therapeutics is common. There is thus a need for improved therapeutics or therapeutic combinations for effective treatment of diseases such as EGFR and/or c-Met positive cancers.

### SUMMARY OF THE INVENTION

In meeting these needs, the present disclosure provides methods of treating an epidermal growth factor receptor (EGFR)-expressing or hepatocyte growth factor receptor (c-Met)-expressing cancer in a subject in need thereof.

The methods of the invention comprise administering to the subject an isolated bispecific anti-EGFR/c-Met antibody comprising a first domain comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and a second domain comprising a HCDR1 of SEQ ID NO: 7, a HCDR2 of SEQ ID NO: 8, a HCDR3 of SEQ ID NO: 9, a LCDR1 of SEQ ID NO: 10, a LCDR2 of SEQ ID NO: 11 and a LCDR3 of SEQ ID NO: 12, at a dose of about 1400-2100 mg, administered once per a 21-day cycle.

In some embodiments, the method further comprises administration of a 3rd generation EGFR tyrosine kinase inhibitor (TKI).

In some embodiments, the 3rd generation EGFR TKI is of formula (I):

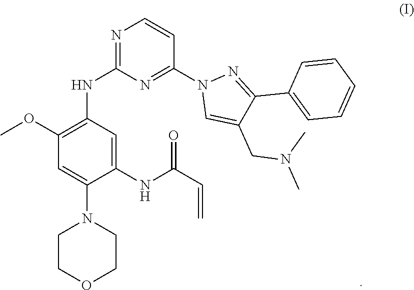 [SMILES: C=CC(=O)Nc1cc(Nc2nccc(-n3cc(CN(C)C)c(-c4ccccc4)n3)n2)c(OC)cc1N1CCOCC1]

In some embodiments, 3rd generation EGFR TKI is administered at a dose of 240 mg daily.

In some embodiments, the starting dose of the bispecific anti-EGFR/c-Met antibody is a split dose given over two consecutive days, wherein
on day one (D1) of the first cycle, the antibody is administered at a dose of about 350 mg, and on day two (D2) of the first cycle, the antibody is administered at a dose of about 1050 mg if the subject weighs less than 80 kg, or
on D1 of the first cycle, the antibody is administered at a dose of about 350 mg, and on D2 of the first cycle, the antibody is administered at a dose of about 1400 mg if the subject weighs 80 kg or more.

In some embodiments, the method further comprises an initial cycle (Cycle 1) comprising administering the bispecific antibody once a week for three weeks at a dose of about 1400 mg if the subject weighs less than 80 kg, or at a dose of about 1750 mg if the subject weighs 80 kg or more.

In some embodiments, the method further comprises a second cycle (Cycle 2) comprising administering the bispecific antibody once in three weeks at a dose of about 1400 mg if the subject weighs less than 80 kg, or at a dose of about 1750 mg if the subject weighs 80 kg or more.

In some embodiments, the method comprises administering the bispecific antibody once every 3 weeks at a dose of about 1750 mg if the subject weighs less than 80 kg, or at a dose of about 2100 mg if the subject weighs 80 kg or more.

In some embodiments, the method further comprises one or more chemotherapeutic agents comprising platinum. In some embodiments, the one or more chemotherapeutic agents comprise pemetrexed and/or carboplatin. In some embodiments, the one or more chemotherapeutic agents are pemetrexed and carboplatin, administered at day 1 of each 21-day cycle. In some embodiments, the one or more chemotherapeutic agents are pemetrexed administered at a dose of about 500 mg/m2 and carboplatin administered to achieve area under the curve (AUC) 5. In some embodiments, the one or more chemotherapeutic agents are pemetrexed administered at a dose of about 500 mg/m2 and carboplatin AUC 5, administered at day 1 of each cycle up to and including cycle four (Cycle 4), and wherein pemetrexed is administered at a dose of about 500 mg/m2 at day 1 of each subsequent cycle.

In some embodiments, the first domain of the bispecific anti-EGFR/c-Met antibody binds EGFR and the second domain binds c-Met.

In some embodiments, the first domain that binds EGFR comprises a heavy chain variable region (VH) of SEQ ID NO: 13 and a light chain variable region (VL) of SEQ ID NO: 14 and the second domain that binds c-Met comprises the VH of SEQ ID NO: 15 and the VL of SEQ ID NO: 16.

In some embodiments, the bispecific anti-EGFR/c-Met antibody is an IgG1 isotype.

In some embodiments, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments, the bispecific anti-EGFR/c-Met antibody has a biantennary glycan structure with a fucose content of between about 1% to about 15%.

In some embodiments, the compound of formula (I) or solvate, hydrate, tautomer, or a pharmaceutically acceptable salt thereof is lazertinib mesylate represented by a compound of formula (II)

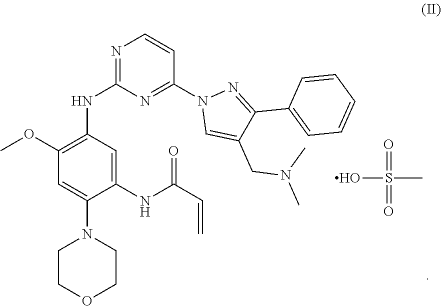

In some embodiments, the compound of formula (I) or solvate, hydrate, tautomer, or a pharmaceutically acceptable salt thereof is N-(5-(4-(4-((dimethylamino)methyl)-3-phenyl-1H-pyrazol-1-yl)pyrimidin-2-ylamino)-4-methoxy-2-morpholinophenyl) acrylamide.

In some embodiments, the EGFR or c-Met expressing cancer is a lung cancer.

In some embodiments, the cancer is a non-small cell lung cancer (NSCLC).

In some embodiments, the cancer is the NSCLC. In some embodiments, the NSCLC is advanced or metastatic EGFR-mutated NSCLC. According to some embodiments, the subject having advanced or metastatic EGFR-mutated NSCLC failed prior treatment with EGFR TKI.

### BRIEF DESCRIPTION OF THE DRAWINGS

FIG. 1 is a diagram depicting overview of the study.

FIG. 2 is a diagram depicting dosing synchronization of amivantamab and chemotherapy for LACP cohort.

FIGS. 3A-C show Ctrough values for amivantamab measured on day 8 of cycle 1 (FIG. 3A), day 15 of cycle 1 (FIG. 3B), and day 22 of cycle 1 (FIG. 3C).

FIGS. 4A-B show Ctrough values for amivantamab measured on day 1 of cycle 2 (FIG. 4A) and day 15 of cycle 2 (FIG. 4B).

FIG. 5 shows the best change from baseline in SoD (sum of leision diameter) of target leisions (%) in patients treated with amivantamab in combination with chemotherapy (carboplatin and pemetrexed).

### DETAILED DESCRIPTION OF THE INVENTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

### Definitions

As used herein, the terms “sequence” and “amino acid sequence” mean a succession or order of amino acids described with a succession of letters using standard nomenclature. An amino acid can comprise unmodified and/or modified amino acid.

As used herein, the terms “individual”, “patient” and “subject”, are used interchangeably to refer to a member of any animal species including, but not limited to, birds, humans and other primates, and other mammals including commercially relevant mammals or animal models such as mice, rats, monkeys, cattle, pigs, horses, sheep, cats, and dogs. Preferably, the subject is a human.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used in a claim herein, the phrase “consisting of” in a clause excludes any element, step, or ingredient not specified in the clause. When used in a clause in a claim herein, the phrase “consisting essentially of” limits the scope of the clause of the claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“About once a week” or “weekly” refers to administration one time over about a one-week period. About a one-week period refers 7 days±two days, i.e., 5 days to 9 days. The dosing frequency of “about once a week” thus can be once every five days, once every six days, once every seven days, once every eight days, or once every nine days.

“About once in three weeks” refers to administration one time over about a three-week period. About a three-week period refers to 21 days±two days, i.e., 19 days to 23 days. The dosing frequence of “about once in three weeks” thus can be once every 19 days, once every 20 days, once every 21 days, once every 22 days or once every 23 days.

“Antagonist” or “inhibitor” refers to a molecule that, when bound to a cellular protein, suppresses at least one reaction or activity that is induced by a natural ligand of the protein. A molecule is an antagonist when the at least one reaction or activity is suppressed by at least about 20%, 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (2), based on the amino acid sequences of their constant domains.

“Antigen binding fragment” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Bispecific anti-EGFR/c-Met antibody” or “bispecific EGFR/c-Met antibody” refers to a bispecific antibody having a first domain that specifically binds EGFR and a second domain that specifically binds c-Met. The domains specifically binding EGFR and c-Met are typically VH/VL pairs. The bispecfic antibody may be, depending on the structure, monovalent, bivalent or multivalent in terms of binding to EGFR and c-Met; i.e., can have one or more domains that bind EGFR and one or more domains that bind c-Met.

“Biological sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids and the like, tissue biopsies, tumor tissue biopsies, tumor tissue samples, fine needle aspirations, surgically resected tissue, organ cultures or cell cultures.

“Complementarity determining regions” (CDR) are antibody regions that bind an antigen. CDRs may be defined using various delineations such as Kabat (Wu et al. (1970) J Exp Med 132:211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196:901-17), IMGT (Lefranc et al. (2003) Dev Comp Immunol 27:55-77) and AbM (Martin and Thornton (1996) J Bmol Biol 263:800-15). The correspondence between the various delineations and variable region numbering are described (see e.g. Lefranc et al. (2003) Dev Comp Immunol 27:55-77; Honegger and Pluckthun, (2001) J Mol Biol 309:657-70; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification

“Cancer” refers to an abnormal growth of cells which tend to proliferate in an uncontrolled way and, in some cases, to metastasize (spread) to other areas of a patient's body.

“Co-administration,” “administration with,” “administration in combination with,” “in combination with”, “combination therapy” or the like, encompass administration of two or more therapeutics to a single patient, and are intended to include treatment regimens in which the therapeutics are administered by the same or different route of administration or at the same or different time.

“Diagnosing” or “diagnosis” refers to methods to determine if a subject is suffering from a given disease or condition or may develop a given disease or condition in the future or is likely to respond to treatment for a prior diagnosed disease or condition, i.e., stratifying a patient population on likelihood to respond to treatment. Diagnosis is typically performed by a physician based on the general guidelines for the disease to be diagnosed or other criteria that indicate a subject is likely to respond to a particular treatment.

“Dosage” refers to the information of the amount of the therapeutic or the drug to be taken by the subject and the frequency of the number of times the therapeutic is to be taken by the subject.

“Dose” refers to the amount or quantity of the therapeutic or the drug to be taken each time.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“EGFR or c-Met expressing cancer” refers to cancer that has detectable expression of EGFR or c-Met or has EGFR or c-Met mutation or amplification. EGFR or c-Met expression, amplification and mutation status can be detected using known methods, such as sequencing, fluorescent in situ hybridization, immunohistochemistry, flow cytometry or western blotting using tumor biopsies or blood samples. Expression can also be detected by sequening from circulating tumor DNA (ctDNA).

“Epidermal growth factor receptor” or “EGFR” refers to the human EGFR (also known as HER1 or ErbB1 (Ullrich et al., Nature 309:418-425, 1984) having the amino acid sequence shown in GenBank accession number NP_005219, as well as naturally occurring variants thereof.

“Hepatocyte growth factor receptor” or “c-Met” as used herein refers to the human c-Met having the amino acid sequence shown in GenBank Accession No: NP_001120972 and natural variants thereof.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”

“Isolated” refers to a homogenous population of molecules (such as synthetic polynucleotides, polypeptides vectors or viruses) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Fucose content” refers to the amount of the fucose monosaccharide within the sugar chain at Asn297 in an antibody preparation.

“Low fucose” or “low fucose content” refers to antibodies with fucose content of about between 1%-15%.

“Normal fucose” or ‘normal fucose content” refers to antibodies with fucose content of over about 50%, typically over about 80% or over about 85%.

“Pharmaceutically acceptable carrier” or “excipient” refers to an ingredient in a pharmaceutical composition, other than the active ingredient, which is nontoxic to a subject. A pharmaceutically acceptable carrier includes, but is not limited to, a buffer, stabilizer or preservative. A pharmaceutically acceptable carrier includes, but is not limited to, a diluent, disintegrant, or glidant; or a diluent, disintegrant, wetting agent, glidant or lubricant.

“Prevent”, “preventing”, “prevention”, or “prophylaxis” of a disease or disorder means preventing that a disorder occurs in subject.

“Recombinant” refers to DNA, antibodies and other proteins that are prepared, expressed, created or isolated by recombinant means when segments from different sources are joined to produce recombinant DNA, antibodies or proteins.

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Solvates” and “hydrates” are solvent addition forms which the compounds of the present invention are able to form, whereby the multicomponent compound contains both the host molecule (e.g., compound of Formula (I) or salt thereof) and guest molecule (water (“hydrate”) or another solvent (“solvate”)) incorporated in the structure.

“Specific binding” or “specifically binds” or “specifically binding” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 5×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The dissociation constant may be measured using known protocols. Antibodies that bind to the antigen or the epitope within the antigen may, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody binds one antigen or one epitope, a bispecific antibody binds two distinct antigens or two distinct epitopes.

“Treatment naïve” in a context of EGFR tyrosine kinase inhibitor (TKI) refers to a subject who has not received EGFR TKI treatment.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“1^{st} generation EGFR tyrosine kinase inhibitor” (1^{st} generation TKI) refers to reversible EGFR inhibitors such as gefitinib and erlotinib, which are effective in first-line treatment of NSCLC harboring EGFR activating mutations such as deletions in exon 19 and exon 21 L858R mutation.

“2^{nd} generation EGFR tyrosine kinase inhibitor” (2^{nd} generation TKI) refers to covalent irreversible EGFR inhibitors such as afatinib and dacomitib which are effective in first-line treatment of NSCLC harboring EGFR activating mutations such as deletions in exon 19 and exon 21 L858R mutation.

“3^{rd} generation EGFR tyrosine kinase inhibitor” (3^{rd} generation TKI) refers to covalent irreversible EGFR inhibitors such as osimertinib and lazertinib which are selective to the EGFR activating mutations, such as deletions in exon 19 and exon 21 L858R, alone or in combination with T790M mutation and have lower inhibitory activity against wild-type EGFR.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### Methods of Therapy

### 1. The Bispecific Anti-EGFR/c-Met Antibody

The bispecific anti-EGFR/c-Met antibody that inhibits both EGFR and c-Met signaling, by blocking ligand-induced activation and by inducing receptor degradation and is described in U.S. Pat. No. 9,593,164, which is incorporated by reference herein. In addition, the presence of high levels of EGFR and c-Met on the surface of tumor cells enables targeting of these cells for destruction by immune effector cells through Fc-mediated effector mechanisms, such as antibody-dependent cellular cytotoxicity (ADCC) and antibody-dependent cellular phagocytosis (ADCP). In some embodiments, the bispecific anti-EGFR/c-Met antibody is amivantamab, also referred to herein as “JNJ-61186372” defined by the following amino acid sequences: the EGFR binding domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6; the c-Met binding domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12; the EGFR binding domain comprises a heavy chain variable domain (VH) of SEQ ID NO: 13 and a light chain variable domain (VL) of SEQ ID NO: 14; the c-Met binding domain comprises the VH of SEQ ID NO: 15 and the VL of SEQ ID NO: 16; the antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

>SEQ ID NO: 1 (HCDR1, EGFR binding arm)
TYGMH
>SEQ ID NO: 2 (HCDR2, EGFR binding arm)
VIWDDGSYKYYGDSVKG
>SEQ ID NO: 3 (HCDR3, EGFR binding arm)
DGITMVRGVMKDYFDY
>SEQ ID NO: 4 (LCDR1, EGFR binding arm)
RASQDISSALV
>SEQ ID NO: 5 (LCDR2, EGFR binding arm)
DASSLES
>SEQ ID NO: 6 (LCDR3, EGFR binding arm)
QQFNSYPLT
>SEQ ID NO: 7 (HCDR1, c-Met binding arm)
SYGIS
>SEQ ID NO: 8 (HCDR2, c-Met binding arm)
WISAYNGYTNYAQKLQG
>SEQ ID NO:9 (HCDR3, c-Met binding arm)
DLRGTNYFDY
>SEQ ID NO: 10 (LCDR1, c-Met binding arm)
RASQGISNWLA
>SEQ ID NO: 11 (LCDR2, c-Met binding arm)
AASSLLS
>SEQ ID NO: 12 (LCDR3, c-Met binding arm)
QQANSFPIT
>SEQ ID NO: 13 (VH, EGFR binding arm)
QVQLVESGGGVVQPGRSLRLSCAASGFTFSTYGMHWVRQAPGKGLEWVAV
IWDDGSYKYYGDSVKGRFTISRDNSKNTLYLQMNSLRAEDTAVYYCARDG
ITMVRGVMKDYFDYWGQGTLVTVSS
>SEQ ID NO: 14 (VL, EGFR binding arm)
AIQLTQSPSSLSASVGDRVTITCRASQDISSALVWYQQKPGKAPKLLIYD
ASSLESGVPSRFSGSESGTDFTLTISSLQPEDFATYYCQQFNSYPLTFGG
GTKVEIK
>SEQ ID NO: 15 (VH, c-Met binding arm)
QVQLVQSGAEVKKPGASVKVSCETSGYTFTSYGISWVRQAPGHGLEWMGW
ISAYNGYTNYAQKLQGRVTMTTDTSTSTAYMELRSLRSDDTAVYYCARDL
RGTNYFDYWGQGTLVTVSS
>SEQ ID NO: 16 (VL, c-Met binding arm)
DIQMTQSPSSVSASVGDRVTITCRASQGISNWLAWFQHKPGKAPKLLIYA
ASSLLSGVPSRFSGSGSGTDFTLTISSLQPEDFATYYCQQANSFPITFGQ
GTRLEIK
>SEQ ID NO: 17 HC1
QVQLVESGGGVVQPGRSLRLSCAASGFTFSTYGMHWVRQAPGKGLEWVAV
IWDDGSYKYYGDSVKGRFTISRDNSKNTLYLQMNSLRAEDTAVYYCARDG
ITMVRGVMKDYFDYWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFLLYSKLTVDKSRWQQGNVFSCSVMHEALHNHYTQKSLS
LSPGK
>SEQ ID NO: 18 LC1
AIQLTQSPSSLSASVGDRVTITCRASQDISSALVWYQQKPGKAPKLLIYD
ASSLESGVPSRFSGSESGTDFTLTISSLQPEDFATYYCQQFNSYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
>SEQ ID NO: 19 HC2
QVQLVQSGAEVKKPGASVKVSCETSGYTFTSYGISWVRQAPGHGLEWMGW
ISAYNGYTNYAQKLQGRVTMTTDTSTSTAYMELRSLRSDDTAVYYCARDL
RGTNYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSRLTVDKSRWQQGNVFSCSVMHEALHNHYTQKSLSLSPGK
>SEQ ID NO: 20 LC2
DIQMTQSPSSVSASVGDRVTITCRASQGISNWLAWFQHKPGKAPKLLIYA
ASSLLSGVPSRFSGSGSGTDFTLTISSLQPEDFATYYCQQANSFPITFGQ
GTRLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In some embodiments, the bispecific anti-EGFR/c-Met antibody is an IgG1 isotype. Some variation exists within the IgG1 constant domain (e.g. well-known allotypes), with variation at positions 214, 356, 358, 422, 431, 435 o 436 (residue numbering according to the EU numbering) (see e.g. IMGT Web resources; IMGT Repertoire (IG and TR); Proteins and alleles; allotypes). The bispecific anti-EGFR/c-Met antibody may be any IgG1 allotype, such as G1m17, G1m3, G1 ml, G1m2, G1m27 or G1m28. The amino acid sequence of an exemplary IgG1 constant domain is shown in SEQ ID NO: 21.

IgG1 constant domain
(SEQ ID NO: 21)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the bispecific anti-EGFR/c-Met antibody comprises the HC1 of SEQ ID NO: 17, the LC1 of SEQ ID NO: 18, the HC2 of SEQ ID NO: 19 and the LC2 of SEQ ID NO: 20.

In some embodiments, the bispecific anti-EGFR/c-Met antibody has a biantennary glycan structure with a fucose content of about between 1% to about 15%.

In some embodiments, the bispecific anti-EGFR/c-Met antibody has a biantennary glycan structure with fucose content of about between 1% to about 15%, for example 15%, 14%, 13%, 12%, 11% 10%, 9%, 8%, 7%, 6%, 5%, 4%, 3%, 2% or 1%.

The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Int Pat. Publ. No. WO2008/077546 2); 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosacharride forms according to hydrophilicity criteria by normal-phase HPLC (GlycoSep N), and separation and quantification of the oligosaccharides by high performance capillary electrophoresis-laser induced fluorescence (HPCE-LIF).

The ability of antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides enhances antibody-dependent cell-mediated cytotoxicity (ADCC) of antibodies via improved FcγRIIIa binding without altering antigen binding or complement dependent cytotoxicity (CDC) activity. Antibodies with reduced fucose content can be made using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2 (4), 2010; Epub ahead of print; PMID: 20562582), application of a rat hybridoma cell line YB2/0 as the host cell line (Shinkawa et al., J Biol Chem 278:3466-3473, 2003), introduction of small interfering RNA specifically against the β 1,6-fucosyltransferase (FUT8) gene (Mori et al., Biotechnol Bioeng 88:901-908, 2004), or coexpression of β-1,4-N-acetylglucosaminyltransferase III and Golgi α-mannosidase II or a potent alpha-mannosidase I inhibitor, kifunensine (Ferrara et al., Biotechnol Bioeng 93:851-861, 2006; Xhou et al., Biotechnol Bioeng 99:652-65, 2008).

### 2. Third Generation EGFR Tyrosine Kinase Inhibitor (TKI)

Lazertinib is a 3^{rd} generation EGFR tyrosine kinase inhibitor (TKI); the structure and synthesis of lazertinib is described in U.S. Pat. No. 9,593,098, which is incorporated by reference herein. The chemical name of the lazertinib free base, which is represented by formula (I) herein, is N-(5-(4-(4-((dimethylamino)methyl)-3-phenyl-1H-pyrazol-1-yl)pyrimidin-2-ylamino)-4-methoxy-2-morpholinophenyl) acrylamide (referred to herein as lazertinib). The mesylate salt of lazertinib may be represented by formula II:

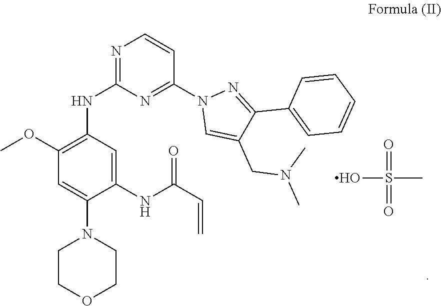

Embodiments of lazertinib (e.g., salts and crystalline forms) are described in PCT/KR2018/004473, which is also incorporated by reference herein.

According to particular embodiments, lazertinib in the form of a free base has little to no effect on wild-type EGFR, and is a highly selective and irreversible EGFR TKI with strong inhibitory activity against the single mutation of T790M and dual mutations; e.g., it targets the activating EGFR mutations del19 and L858R, as well as the T790M mutation. In one aspect of the invention, the mutation may be delE746-A750, L858R, or T790M, and it may be dual mutations selected from delE746-A750/T790M or L858R/T790M.

The disclosure provides methods of treating an EGFR-expressing or c-Met-expressing cancer in a subject in need thereof, comprising administering to the subject a combination therapy, wherein the combination therapy comprises a bispecific antibody, a third generation EGFR TKI of formula (I), or solvate, hydrate, tautomer, or a pharmaceutically acceptable salt thereof, and one or more chemotherapeutic agents.

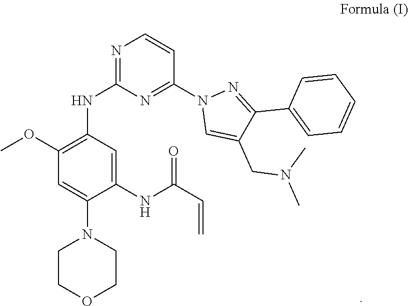 [SMILES: C=CC(=O)Nc1cc(Nc2nccc(-n3cc(CN(C)C)c(-c4ccccc4)n3)n2)c(OC)cc1N1CCOCC1]

In each embodiment, the bispecific anti-EGFR/c-Met antibody and the lazertinib compound, or solvate, hydrate, tautomer, or a pharmaceutically acceptable salt thereof, may be administered at the same time (e.g., as part of the same pharmaceutical composition, or in separate pharmaceutical compositions) or at different times, as described herein.

Pharmaceutically acceptable salt forms include pharmaceutically acceptable acidic/anionic or basic/cationic salts. Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine and triethanolamine salts.

A pharmaceutically acceptable acid salt is formed by reaction of the free base form of a compound of Formula (I) with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid. A pharmaceutically acceptable acid addition salt of a compound of Formula (I) can comprise or be, for example, a hydrobromide, hydrochloride, sulfate, nitrate, phosphate, succinate, maleate, formarate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g., 2-naphthalenesulfonate) or hexanoate salt.

The free acid or free base forms of the compound of formula (I) may be prepared from the corresponding base addition salt or acid addition salt form, respectively. For example a compound of the invention in an acid addition salt form may be converted to the corresponding free base form by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

### 3. Chemotherapeutic Agents

The disclosed methods include a combination therapy comprising administering one or more chemotherapeutic agents as part of the combination therapy. The chemotherapeutic agents comprise platinum-based chemotherapueitcs. The methods can comprise administering platinum-based doublet chemotherapy. For example, platinum-based doublet chemotherapy can comprise administering intravenous pemetrexed (500 mg per square meter of body-surface area) plus either carboplatin (target area under the curve, 5 [AUC5]) or cisplatin (75 mg per square meter). The administering of the one or more chemotherapeutic agents can comprise administering pemetrexed and carboplatin once every 21-cycle, for up to four cycles, and then maintenance of pemetrexed at once every 21-cycle thereafter.

### 4. Doses and 21-Day Cycle

Disclosed is a method of treating of an EGFR-expressing or hepatocyte growth factor receptor (c-Met)-expressing cancer in a subject in need thereof. In some embodiments, the method comprises administering to the subject an isolated bispecific anti-EGFR/c-Met antibody, wherein the method comprises administering the antibody once per 21-day cycle for one or more cycles. In some embodiments, the method comprise administering to the subject a combination therapy comprising 1) a bispecific antibody comprising an isolated bispecific anti-EGFR/c-Met antibody, optionally 2) third generation EGFR tyrosine kinase inhibitor (TKI), and optionally 3) one or more chemotherapeutic agents. In some embodiments, the combination therapy is administered in 21-day cycles.

In some embodiments, the bispecific antibody is administered in three or more 21-day cycles at a starting dose of about about 1400 mg to the subject weighing less than 80 kg, or about 1750 mg to the subject weighing 80 kg or more. In some embodiments, the starting dose is a split dose given over two consecutive days. For example, on day one (D1) of the first cycle, the antibody is administered at a dose of about 350 mg, and on day two (D2) of the first cycle, the antibody is administered at a dose of about 1050 mg if the subject weighs less than 80 kg for the full starting dose of 1400 mg. Alterantively, on D1 of the first cycle, the antibody is administered at a dose of about 350 mg, and on D2 of the first cycle, the antibody is administered at a dose of about 1400 mg if the subject weighs 80 kg or more for the full starting dose of 1750 mg.

In the first cycle (Cycle 1) of the three or more cycles, the methods can comprise administering the bispecific antibody once a week at a dose of about 1400 mg if the subject weighs less than 80 kg, or at a dose of about 1750 mg if the subject weighs 80 kg or more.

In the second cycle (Cycle 2) of the three or more cycles, the methods can comprise administering the bispecific antibody once at a dose of about 1400 mg if the subject weighs less than 80 kg, or at a dose of about 1750 mg if the subject weighs 80 kg or more.

In the third cycle (Cycle 3) of the three or more cycles, the methods can comprise administering the bispecific antibody once at a dose of about 1750 mg if the subject weighs less than 80 kg, or at a dose of about 2100 mg if the subject weighs 80 kg or more.

In cycles subsequent to Cycle 3, the methods can comprise administering the bispecific antibody once at a dose of about 1750 mg if the subject weighs less than 80 kg, or at a dose of about 2100 mg if the subject weighs 80 kg or more.

In some embodiments, the administering of the bispecific antibody is by intravenous administration. In some embodiments, the administering of the bispecific antibody is by subcutaneous administration.

In some embodiments, the bispecific anti-EGFR/c-Met antibody is administered subcutaneously at a dose of between about 1400 mg to about 2240 mg. In some embodiments, the bispecific anti-EGFR/c-Met antibody is administered subcutaneously at a dose of about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1575 mg, about 1580 mg, about 1590 mg, about 1600 mg, about 1610 mg, 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, 1890 mg, about 1900 mg, about 1910 mg, about 1920 mg, about 1930 mg, about 1940 mg, about 1950 mg, about 1960 mg, about 1970 mg, about 1980 mg, about 1990 mg, about 2000 mg, about 2010 mg, about 2020 mg, about 2030 mg, about 2040 mg, about 2050 mg, about 2060 mg, about 2070 mg, about 2080 mg, about 2090 mg, about 2100 mg, about 2110 mg, about 2120 mg, about 2150 mg, about 2200 mg, about 2210 mg, about 2220 mg, about 2230 mg, about 2240 mg, about 2250 mg, about 2260 mg, about 2270 mg, about 2280 mg, about 2290 mg, or about 2300 mg.

In some embodiments, the bispecific anti-EGFR/c-Met antibody is administered subcutaneously at a dose of about 1400 mg, comprising administration once every 21 days. In some embodiments, the bispecific anti-EGFR/c-Met antibody is administered subcutaneously at a dose of about 1575 mg, comprising administration once every 21 days. In some embodiments, the bispecific anti-EGFR/c-Met antibody is administered subcutaneously at a dose of about 1600 mg, comprising administration once every 21 days. In some embodiments, the bispecific anti-EGFR/c-Met antibody is administered subcutaneously at a dose of about 2100 mg, comprising administration once every 21 days. In some embodiments, the bispecific anti-EGFR/c-Met antibody is administered subcutaneously at a dose of about 2240 mg, comprising administration once every 21 days.

In some embodiments, the third generation EGFR TKI is administered to the subject daily during each of the three or more cycles.

In some embodiments, the third generation EGFR TKI is administered to the subject daily at a dose between about 80 mg and about 240 mg during each of the three or more cycles. In some embodiments, the third generation EGFR TKI is administered to the subject daily at a dose of about 80 mg. In some embodiments, the third generation EGFR TKI is administered to the subject daily at a dose of about 160 mg. In some embodiments, the third generation EGFR TKI is administered to the subject daily at a dose of about 240 mg.

The one or more chemotherapeutic agents comprise platinum, optionally wherein the chemotherapeutic agent comprises carboplatin, preferably wherein the chemotherapeutic agents comprise pemetrexed and carboplatin.

In some embodiments, the one or more chemotherapeutic agents are pemetrexed and carboplatin, administered at day 1 of each cycle, up to and including cycle four (Cycle 4).

In some embodiments, the one or more chemotherapeutic agents are pemetrexed administered at a dose of about 500 mg/m2 and carboplatin administered to achieve area under the curve (AUC) 5.

In some embodiments, the one or more chemotherapeutic agents are pemetrexed administered at a dose of about 500 mg/m2 and carboplatin AUC 5, administered at day 1 of each cycle up to and including cycle four (Cycle 4), and wherein pemetrexed is administered at a dose of about 500 mg/m2 at day 1 of each subsequent cycle.

### Methods of Treating an EGFR-Expressing or c-Met-Expressing Cancer

The disclosed methods are useful for treating EGFR or c-Met expressing cancer in the subject. The EGFR or c-Met expressing cancer can comprise associated with a wild-type EGFR, an EGFR mutation, an EGFR gene amplification, increased levels of circulating HGF, a wild-type c-Met, a c-Met mutation, a c-Met gene amplification or a mutant KRAS.

In some embodiments, the EGFR mutation is E709K, L718Q, L718V, G719A, G719X, G724X, G724S, 1744T, E746K, L747S, E749Q, A750P, A755V, V765M, C775Y, T790M, L792H, L792V, G796S, G796R, G796C, C797S, T854I, L858P, L858R, L861X, delE746-A750, delE746_T751InsKV, delE746_A750InsHS, delE746_T751InsFPT, delE746_T751InsL, delE746_S752InsIP, delE746_P753InsMS, delE746_T751InsA, delE746_T751InsAPT, delE746_T751InsVA, delE746_S752InsV, delE746_P753InsVS, delE746_K754InsGG, delE746_E749, delE746_E749InsP, delL747_E749, delL747_A750InsP, delL747_T751InsP, delL747_T751InsN, delL747_S752InsPT, delL747_P753InsNS, delL747_S752InsPI, delL747_S752, delL747_P753InsS, delL747_K754, dekL747_T751InsS, dekL747_T751, delL747_P753InsS, delA750_I759InsPT, delT751_I759InsT, delS752_I759, delT751_I759InsN, delT751_D761InsNLY, delS752_I759, delR748-P753, delL747-P753insS, delL747-T751, M766_A767InsA, S768_V769InsSVA, P772_H773InsNS, D761_E762InsX₁₋₇, A763_Y764InsX₁₋₇, Y764_Y765 InsX₁₋₇, M766_A767InsX₁₋₇, A767_V768 InsX₁₋₇, S768_V769 InsX₁₋₇, V769_D770 InsX₁₋₇, D770_N771 InsX₁₋₇, N771_P772 InsX₁₋₇, P772_H773 InsX₁₋₇, H773_V774 InsX₁₋₇, V774_C775 InsX₁₋₇, one or more deletions in EGFR exon 20, or one or more insertions in EGFR exon 20, one or more deletions in EGFR exon 19, or one or more insertions in EGFR exon 19, or any combination thereof, wherein X is any amino acids.

In some embodiments, the EGFR mutation is the one or more deletions in exon 19 or L858R, or any combination thereof.

In some embodiments, the c-Met mutation is c-Met exon 14 skipping mutation.

In some embodiments, the mutant KRAS has a G12V, G12C or G12A substitution.

In some embodiments, the subject has been diagnosed with the EGFR mutation prior to administering the combination therapy.

In some embodiments, the subject has a newly diagnosed EGFR or c-Met expressing cancer.

In some embodiments, the subject is EGFR tyrosine kinase inhibitor (TKI) treatment naïve.

In some embodiments, subject is resistant to or relapsed following treatment with erlotinib or gefitinib.

In some embodiments, the subject is resistant to or relapsed following treatment with afatinib.

In some embodiments, the subject is resistant to or relapsed following treatment with osimertinib.

In some embodiments, the EGFR or c-Met expressing cancer is a non-small cell lung cancer (NSCLC), an epithelial cell cancer, a breast cancer, an ovarian cancer, a lung cancer, a squamous cell lung cancer, a lung adenocarcinoma, a small cell lung cancer, a colorectal cancer, an anal cancer, a prostate cancer, a kidney cancer, a bladder cancer, a head and neck cancer, a pharynx cancer, a cancer of the nose, a pancreatic cancer, a skin cancer, an oral cancer, a cancer of the tongue, an esophageal cancer, a vaginal cancer, a cervical cancer, a cancer of the spleen, a testicular cancer, a gastric cancer, a cancer of the thymus, a colon cancer, a thyroid cancer, a liver cancer, a hepatocellular carcinoma (HCC) or sporadic or hereditary papillary renal cell carcinoma (PRCC).

In some embodiments, the cancer is a NSCLC.

### 1. Treating an EGFR-Expressing or c-Met-Expressing Cancer in Subjects Weighing Less than 80 kg

The methods of treating an EGFR-expressing or c-Met-expressing cancer in a subject weighing less than 80 kg can comprise
administering to the subject in three or more 21-day cycles a combination therapy comprising:
(a) pemetrexed and carboplatin,
(b) a bispecific antibody comprising an isolated bispecific anti-EGFR/c-Met antibody comprising a first domain comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and a second domain comprising a HCDR1 of SEQ ID NO: 7, a HCDR2 of SEQ ID NO: 8, a HCDR3 of SEQ ID NO: 9, a LCDR1 of SEQ ID NO: 10, a LCDR2 of SEQ ID NO: 11 and a LCDR3 of SEQ ID NO: 12, and
(c) a third generation EGFR tyrosine kinase inhibitor (TKI) of formula (I)

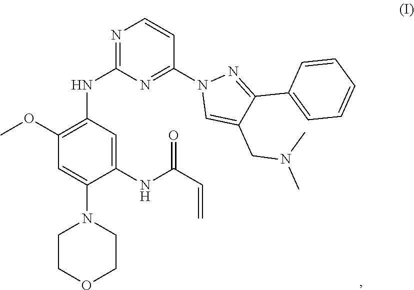 [SMILES: C=CC(=O)Nc1cc(Nc2nccc(-n3cc(CN(C)C)c(-c4ccccc4)n3)n2)c(OC)cc1N1CCOCC1]

or solvate, hydrate, tautomer, or a pharmaceutically acceptable salt thereof,
wherein the three or more 21-day cycles comprise Cycle 1, Cycle 2, and Cycle 3, and
wherein the administering comprises:
administering on day 1 of Cycle 1 the bispecific antibody at about 350 mg, pemetrexed at 500 mg/m2, and carboplatin to achieve AUC 5;
administering on day 2 of Cycle 1 the bispecific antibody at about 1050 mg;
administering on day 8 of Cycle 1 the bispecific antibody at about 1400 mg;
administering on day 15 of Cycle 1 the bispecific antibody at about 1400 mg;
administering on day 1 of Cycle 2 the bispecific antibody at about 1400 mg, pemetrexed at 500 mg/m2, and carboplatin to achieve AUC 5;
administering on day 1 of Cycle 3 the bispecific antibody at about 1750 mg, pemetrexed at 500 mg/m2, and carboplatin to achieve AUC 5; and
wherein the third generation EGFR tyrosine kinase inhibitor (TKI) is administered daily at a dose between about 80 mg and about 240 mg.

In some embodiments, the administering the bispecific antibody is by intravenous administration. In some embodiments, the administering of the bispecific antibody is by subcutaneous administration.

In some embodiments, the bispecific anti-EGFR/c-Met antibody is administered subcutaneously at a dose of between about 1400 mg to about 2240 mg. In some embodiments, the bispecific anti-EGFR/c-Met antibody is administered subcutaneously at a dose of about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1575 mg, about 1580 mg, about 1590 mg, about 1600 mg, about 1610 mg, 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, 1890 mg, about 1900 mg, about 1910 mg, about 1920 mg, about 1930 mg, about 1940 mg, about 1950 mg, about 1960 mg, about 1970 mg, about 1980 mg, about 1990 mg, about 2000 mg, about 2010 mg, about 2020 mg, about 2030 mg, about 2040 mg, about 2050 mg, about 2060 mg, about 2070 mg, about 2080 mg, about 2090 mg, about 2100 mg, about 2110 mg, about 2120 mg, about 2150 mg, about 2200 mg, about 2210 mg, about 2220 mg, about 2230 mg, about 2240 mg, about 2250 mg, about 2260 mg, about 2270 mg, about 2280 mg, about 2290 mg, or about 2300 mg.

In some embodiments, the bispecific anti-EGFR/c-Met antibody is administered subcutaneously at a dose of about 1400 mg, comprising administration once every 21 days. In some embodiments, the bispecific anti-EGFR/c-Met antibody is administered subcutaneously at a dose of about 1575 mg, comprising administration once every 21 days. In some embodiments, the bispecific anti-EGFR/c-Met antibody is administered subcutaneously at a dose of about 1600 mg, comprising administration once every 21 days. In some embodiments, the bispecific anti-EGFR/c-Met antibody is administered subcutaneously at a dose of about 2100 mg, comprising administration once every 21 days. In some embodiments, the bispecific anti-EGFR/c-Met antibody is administered subcutaneously at a dose of about 2240 mg, comprising administration once every 21 days.

In some embodiments, carboplatin is administered for not more than four cycles.

In some embodiments, the third generation EGFR TKI is administered at a dose of about 80 mg, about 160 mg, or about 240 mg.

In some embodiments, the third generation EGFR TKI is administered no more than about 2 hours prior to administering the bispecific antibody.

### 2. Treating an EGFR-Expressing or c-Met-Expressing Cancer in Subjects Weighing 80 kg or More

The methods of treating an EGFR-expressing or c-Met expressing cancer in a subject weighing 80 kg or more can comprise
administering to the subject in three or more 21-day cycles a combination therapy comprising:
(a) pemetrexed and carboplatin,
(b) a bispecific antibody comprising an isolated bispecific anti-EGFR/c-Met antibody comprising a first domain comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and a second domain comprising a HCDR1 of SEQ ID NO: 7, a HCDR2 of SEQ ID NO: 8, a HCDR3 of SEQ ID NO: 9, a LCDR1 of SEQ ID NO: 10, a LCDR2 of SEQ ID NO: 11 and a LCDR3 of SEQ ID NO: 12, and
(c) a third generation EGFR tyrosine kinase inhibitor (TKI) of formula (I)

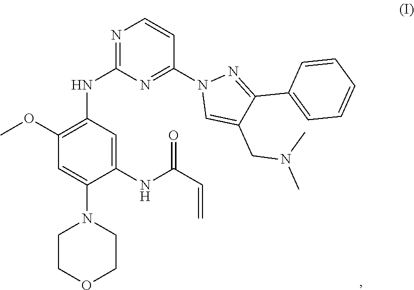 [SMILES: C=CC(=O)Nc1cc(Nc2nccc(-n3cc(CN(C)C)c(-c4ccccc4)n3)n2)c(OC)cc1N1CCOCC1]

or solvate, hydrate, tautomer, or a pharmaceutically acceptable salt thereof,
wherein the three or more 21-day cycles comprise Cycle 1, Cycle 2, and Cycle 3, and
wherein the administering comprises:
administering on day 1 of Cycle 1 the bispecific antibody at about 350 mg, pemetrexed at 500 mg/m2, and carboplatin to achieve AUC 5;
administering on day 2 of Cycle 1 the bispecific antibody at about 1400 mg;
administering on day 8 of Cycle 1 the bispecific antibody at about 1750 mg;
administering on day 15 of Cycle 1 the bispecific antibody at about 1750 mg;
administering on day 1 of Cycle 2 the bispecific antibody at about 1750 mg, pemetrexed at 500 mg/m2, and carboplatin to achieve AUC 5;
administering on day 1 of Cycle 3 the bispecific antibody at about 2100 mg, pemetrexed at 500 mg/m2, and carboplatin to achieve AUC 5; and
wherein the third generation EGFR tyrosine kinase inhibitor (TKI) is administered daily at a dose between about 80 mg and about 240 mg.

In some embodiments, the administering the bispecific antibody is by intravenous administration. In some embodiments, the administering of the bispecific antibody is by subcutaneous administration.

In some embodiments, the bispecific anti-EGFR/c-Met antibody is administered subcutaneously at a dose of between about 1400 mg to about 2240 mg. In some embodiments, the bispecific anti-EGFR/c-Met antibody is administered subcutaneously at a dose of about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1575 mg, about 1580 mg, about 1590 mg, about 1600 mg, about 1610 mg, 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, 1890 mg, about 1900 mg, about 1910 mg, about 1920 mg, about 1930 mg, about 1940 mg, about 1950 mg, about 1960 mg, about 1970 mg, about 1980 mg, about 1990 mg, about 2000 mg, about 2010 mg, about 2020 mg, about 2030 mg, about 2040 mg, about 2050 mg, about 2060 mg, about 2070 mg, about 2080 mg, about 2090 mg, about 2100 mg, about 2110 mg, about 2120 mg, about 2150 mg, about 2200 mg, about 2210 mg, about 2220 mg, about 2230 mg, about 2240 mg, about 2250 mg, about 2260 mg, about 2270 mg, about 2280 mg, about 2290 mg, or about 2300 mg.

In some embodiments, the bispecific anti-EGFR/c-Met antibody is administered subcutaneously at a dose of about 1400 mg, comprising administration once every 21 days. In some embodiments, the bispecific anti-EGFR/c-Met antibody is administered subcutaneously at a dose of about 1575 mg, comprising administration once every 21 days. In some embodiments, the bispecific anti-EGFR/c-Met antibody is administered subcutaneously at a dose of about 1600 mg, comprising administration once every 21 days. In some embodiments, the bispecific anti-EGFR/c-Met antibody is administered subcutaneously at a dose of about 2100 mg, comprising administration once every 21 days. In some embodiments, the bispecific anti-EGFR/c-Met antibody is administered subcutaneously at a dose of about 2240 mg, comprising administration once every 21 days.

In some embodiments, carboplatin is administered for not more than four cycles.

In some embodiments, the third generation EGFR TKI is administered at a dose of about 80 mg, about 160 mg, or about 240 mg.

In some embodiments, the third generation EGFR TKI is administered no more than about 2 hours prior to administering the bispecific antibody.

### EMBODIMENTS

1. A method of treating an epidermal growth factor receptor (EGFR)-expressing or hepatocyte growth factor receptor (c-Met)-expressing cancer in a subject in need thereof, comprising administering to the subject an isolated bispecific anti-EGFR/c-Met antibody comprising a first domain comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and a second domain comprising a HCDR1 of SEQ ID NO: 7, a HCDR2 of SEQ ID NO: 8, a HCDR3 of SEQ ID NO: 9, a LCDR1 of SEQ ID NO: 10, a LCDR2 of SEQ ID NO: 11 and a LCDR3 of SEQ ID NO: 12, at a dose of about 1400-2100 mg, administered once per a 21-day cycle.
2. The method of embodiment 1, wherein the method further comprises administration of a 3^{rd} generation EGFR tyrosine kinase inhibitor (TKI).
3. The method of embodiment 2, wherein the 3^{rd} generation EGFR TKI is of formula (I):

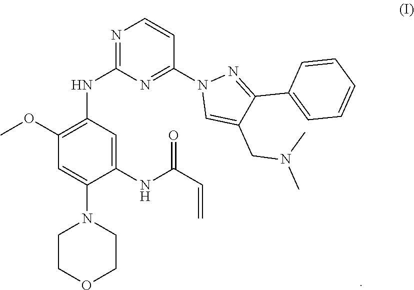 [SMILES: C=CC(=O)Nc1cc(Nc2nccc(-n3cc(CN(C)C)c(-c4ccccc4)n3)n2)c(OC)cc1N1CCOCC1]

4. The method of embodiment 3, wherein the 3^{rd} generation EGFR TKI is administered at a dose of 240 mg daily.
5. The method of embodiment 1, wherein the starting dose is a split dose given over two consecutive days, wherein on day one (D1) of the first cycle, the antibody is administered at a dose of about 350 mg, and on day two (D2) of the first cycle, the antibody is administered at a dose of about 1050 mg if the subject weighs less than 80 kg, or on D1 of the first cycle, the antibody is administered at a dose of about 350 mg, and on D2 of the first cycle, the antibody is administered at a dose of about 1400 mg if the subject weighs 80 kg or more.
6. The method of embodiment 1, wherein the method further comprises an initial cycle (Cycle 1) comprising administering the bispecific antibody once a week for three weeks at a dose of about 1400 mg if the subject weighs less than 80 kg, or at a dose of about 1750 mg if the subject weighs 80 kg or more.
7. The method of embodiment 6, wherein the method further comprises a second cycle (Cycle 2) comprising administering the bispecific antibody once in three weeks at a dose of about 1400 mg if the subject weighs less than 80 kg, or at a dose of about 1750 mg if the subject weighs 80 kg or more.
8. The method of embodiment 1, wherein the method comprises administering the bispecific antibody once every 3 weeks at a dose of about 1750 mg if the subject weighs less than 80 kg, or at a dose of about 2100 mg if the subject weighs 80 kg or more.
9. The method of embodiment 1, wherein the method further comprises one or more chemotherapeutic agents comprising platinum.
10. The method of embodiment 9, wherein the one or more chemotherapeutic agents comprise pemetrexed and/or carboplatin.
11. The method of embodiment 9, wherein the one or more chemotherapeutic agents are pemetrexed and carboplatin, administered at day 1 of each 21-day cycle.
12. The method of embodiment 9, wherein the one or more chemotherapeutic agents are pemetrexed administered at a dose of about 500 mg/m2 and carboplatin administered to achieve area under the curve (AUC) 5.
13. The method of embodiment 9, wherein the one or more chemotherapeutic agents are pemetrexed administered at a dose of about 500 mg/m2 and carboplatin AUC 5, administered at day 1 of each cycle up to and including cycle four (Cycle 4), and wherein pemetrexed is administered at a dose of about 500 mg/m2 at day 1 of each subsequent cycle.
14. A method of treating an epidermal growth factor receptor (EGFR)-expressing or hepatocyte growth factor receptor (c-Met)-expressing cancer in a subject weighing less than 80 kg, the method comprising
administering to the subject in three or more 21-day cycles a combination therapy comprising:
(a) pemetrexed and carboplatin,
(b) a bispecific antibody comprising an isolated bispecific anti-EGFR/c-Met antibody comprising a first domain comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and a second domain comprising a HCDR1 of SEQ ID NO: 7, a HCDR2 of SEQ ID NO: 8, a HCDR3 of SEQ ID NO: 9, a LCDR1 of SEQ ID NO: 10, a LCDR2 of SEQ ID NO: 11 and a LCDR3 of SEQ ID NO: 12, and
(c) a third generation EGFR tyrosine kinase inhibitor (TKI) of formula (I)

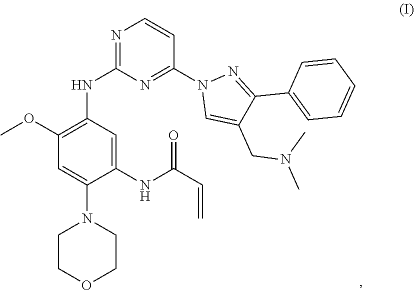 [SMILES: C=CC(=O)Nc1cc(Nc2nccc(-n3cc(CN(C)C)c(-c4ccccc4)n3)n2)c(OC)cc1N1CCOCC1]

or solvate, hydrate, tautomer, or a pharmaceutically acceptable salt thereof,
wherein the three or more 21-day cycles comprise Cycle 1, Cycle 2, and Cycle 3, and
wherein the administering comprises:
administering on day 1 of Cycle 1 the bispecific antibody at about 350 mg, pemetrexed at 500 mg/m2, and carboplatin to achieve AUC 5;
administering on day 2 of Cycle 1 the bispecific antibody at about 1050 mg;
administering on day 8 of Cycle 1 the bispecific antibody at about 1400 mg;
administering on day 15 of Cycle 1 the bispecific antibody at about 1400 mg;
administering on day 1 of Cycle 2 the bispecific antibody at about 1400 mg, pemetrexed at 500 mg/m2, and carboplatin to achieve AUC 5;
administering on day 1 of Cycle 3 the bispecific antibody at about 1750 mg, pemetrexed at 500 mg/m2, and carboplatin to achieve AUC 5; and
wherein the third generation EGFR tyrosine kinase inhibitor (TKI) is administered daily at a dose between about 80 mg and about 240 mg.
15. The method of embodiment 14, wherein administering the bispecific antibody is by (a) intravenous administration or (b) subcutaneous administration.
16. The method of embodiment 14 or 15, wherein carboplatin is administered for not more than four cycles.
17. The method of any one of embodiments 14-16, wherein the third generation EGFR tyrosine kinase inhibitor (TKI) is administered at a dose of about 240 mg.
18. The method of any one of embodiments 14-17, wherein the third generation EGFR tyrosine kinase inhibitor (TKI) is administered no more than about 2 hours prior to administering the bispecific antibody.
19. A method of treating an epidermal growth factor receptor (EGFR)-expressing or hepatocyte growth factor receptor (c-Met) expressing cancer in a subject weighing 80 kg or more, the method comprising
administering to the subject in three or more 21-day cycles a combination therapy comprising:
(a) pemetrexed and carboplatin,
(b) a bispecific antibody comprising an isolated bispecific anti-EGFR/c-Met antibody comprising a first domain comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and a second domain comprising a HCDR1 of SEQ ID NO: 7, a HCDR2 of SEQ ID NO: 8, a HCDR3 of SEQ ID NO: 9, a LCDR1 of SEQ ID NO: 10, a LCDR2 of SEQ ID NO: 11 and a LCDR3 of SEQ ID NO: 12, and
(c) a third generation EGFR tyrosine kinase inhibitor (TKI) of formula (I)

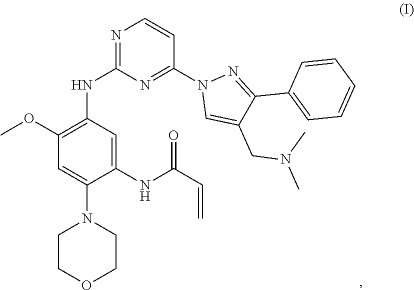 [SMILES: C=CC(=O)Nc1cc(Nc2nccc(-n3cc(CN(C)C)c(-c4ccccc4)n3)n2)c(OC)cc1N1CCOCC1]

or solvate, hydrate, tautomer, or a pharmaceutically acceptable salt thereof,
wherein the three or more 21-day cycles comprise Cycle 1, Cycle 2, and Cycle 3, and
wherein the administering comprises:
administering on day 1 of Cycle 1 the bispecific antibody at about 350 mg, pemetrexed at 500 mg/m2, and carboplatin to achieve AUC 5;
administering on day 2 of Cycle 1 the bispecific antibody at about 1400 mg;
administering on day 8 of Cycle 1 the bispecific antibody at about 1750 mg;
administering on day 15 of Cycle 1 the bispecific antibody at about 1750 mg;
administering on day 1 of Cycle 2 the bispecific antibody at about 1750 mg, pemetrexed at 500 mg/m2, and carboplatin to achieve AUC 5;
administering on day 1 of Cycle 3 the bispecific antibody at about 2100 mg, pemetrexed at 500 mg/m2, and carboplatin to achieve AUC 5; and
wherein the third generation EGFR tyrosine kinase inhibitor (TKI) is administered daily at a dose between about 80 mg and about 240 mg.
20. The method of embodiment 19, wherein administering the bispecific antibody is by (a) intravenous administration or (b) subcutaneous administration.
21. The method of embodiment 19 or 20, wherein carboplatin is administered for not more than four cycles.
22. The method of any one of embodiments 19-21, wherein the third generation EGFR tyrosine kinase inhibitor (TKI) is administered at a dose of about 240 mg.
23. The method of any one of embodiments 19-22, wherein the third generation EGFR tyrosine kinase inhibitor (TKI) is administered no more than about 2 hours prior to administering the bispecific antibody.
24. The method of any one of embodiments 1-23, wherein the first domain of the bispecific anti-EGFR/c-Met antibody binds EGFR and the second domain binds c-Met.
25. The method of embodiment 24, wherein the first domain that binds EGFR comprises a heavy chain variable region (VH) of SEQ ID NO: 13 and a light chain variable region (VL) of SEQ ID NO: 14 and the second domain that binds c-Met comprises the VH of SEQ ID NO: 15 and the VL of SEQ ID NO: 16.
26. The method of any one of embodiments 1 to 25, wherein the bispecific anti-EGFR/c-Met antibody is an IgG1 isotype.
27. The method of any one of embodiments 1 to 26, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.
28. The method of any one of embodiments 1 to 27, wherein the bispecific anti-EGFR/c-Met antibody has a biantennary glycan structure with a fucose content of between about 1% to about 15%.
29. The method of any one of embodiments 1 to 28, wherein the compound of formula (I) or solvate, hydrate, tautomer, or a pharmaceutically acceptable salt thereof is lazertinib mesylate. 30. The method of embodiments 2 to 29, wherein the compound of formula (I) or solvate, hydrate, tautomer, or a pharmaceutically acceptable salt thereof is lazertinib mesylate represented by a compound of formula (II)
31.

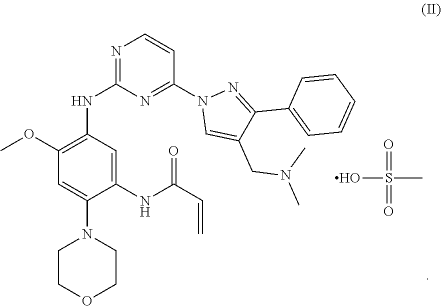

32. The method of any one of embodiments 1 to 30, wherein the EGFR or c-Met expressing cancer is associated with a wild-type EGFR, an EGFR mutation, an EGFR gene amplification, increased levels of circulating HGF, a wild-type c-Met, a c-Met mutation, a c-Met gene amplification or a mutant KRAS.
33. The method of embodiment 31, wherein the EGFR mutation is E709K, L718Q, L718V, G719A, G719X, G724X, G724S, I744T, E746K, L747S, E749Q, A750P, A755V, V765M, C775Y, T790M, L792H, L792V, G796S, G796R, G796C, C797S, T854I, L858P, L858R, L861X, delE746-A750, delE746_T751InsKV, delE746_A750InsHS, delE746_T751InsFPT, delE746_T751InsL, delE746_S752InsIP, delE746_P753InsMS, delE746_T751InsA, delE746_T751InsAPT, delE746_T751InsVA, delE746_S752InsV, delE746_P753InsVS, delE746_K754InsGG, delE746_E749, delE746_E749InsP, delL747_E749, delL747_A750InsP, delL747_T751InsP, delL747_T751InsN, delL747_S752InsPT, delL747_P753InsNS, delL747_S752InsPI, delL747_S752, delL747_P753InsS, delL747_K754, dekL747_T751InsS, dekL747_T751, delL747_P753InsS, delA750_I759InsPT, delT751_I759InsT, delS752_I759, delT751_I759InsN, delT751_D761InsNLY, delS752_I759, delR748-P753, delL747-P753insS, delL747-T751, M766_A767InsA, S768_V769InsSVA, P772_H773InsNS, D761_E762InsX₁₋₇, A763_Y764InsX₁₋₇, Y764_Y765 InsX₁₋₇, M766_A767InsX₁₋₇, A767_V768 InsX₁₋₇, S768_V769 InsX₁₋₇, V769_D770 InsX₁₋₇, D770_N771 InsX₁₋₇, N771_P772 InsX₁₋₇, P772_H773 InsX₁₋₇, H773_V774 InsX₁₋₇, V774_C775 InsX₁₋₇, one or more deletions in EGFR exon 20, or one or more insertions in EGFR exon 20, one or more deletions in EGRF exon 19, or one or more insertions in EGFR exon 19, or any combination thereof, wherein X is any amino acids.
34. The method of embodiment 31 or 32, wherein the EGFR mutation is the one or more deletions in exon 19 or L858R, or any combination thereof.
35. The method of embodiment 31, wherein the c-Met mutation is c-Met exon 14 skipping mutation.
36. The method of embodiment 31, wherein the mutant KRAS has a G12V, G12C or G12A substitution.
37. The method of any one of embodiments 1 to 35, wherein the subject has been diagnosed with the EGFR mutation prior to administering the bi-specific antibody.
38. The method of any one of embodiments 1 to 36, wherein the subject has a newly diagnosed EGFR or c-Met expressing cancer.
39. The method of any one of embodiments 1 to 37, wherein the subject is EGFR tyrosine kinase inhibitor (TKI) treatment naïve.
40. The method of any one of embodiments 1 to 37, wherein the subject has progressed on or after an EGFR-TKI treatment.
41. The method of embodiment 39, wherein the subject is resistant to or relapsed following treatment with erlotinib, gefitinib, lapatinib, vandetanib, afatinib, osimertinib, poziotinib, criotinib, cabozantinib, capmatinib, axitinib, lenvatinib, nintedanib, regorafenib, pazopanib, sorafenib or sunitinib.
42. The method of embodiment 39, wherein the subject is resistant to or relapsed following treatment with osimertinib.
43. The method of any one of embodiments 1 to 41, wherein the EGFR or c-Met expressing cancer is a lung cancer.
44. The method of embodiment 42, wherein the cancer is the NSCLC.
45. A method of treating an epidermal growth factor receptor (EGFR)-expressing or hepatocyte growth factor receptor (c-Met)-expressing cancer in a subject weighing less than 80 kg, the method comprising
administering to the subject in three or more 21-day cycles a combination therapy comprising:
(a) pemetrexed and carboplatin,
(b) a bispecific antibody comprising an isolated bispecific anti-EGFR/c-Met antibody comprising a first domain comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and a second domain comprising a HCDR1 of SEQ ID NO: 7, a HCDR2 of SEQ ID NO: 8, a HCDR3 of SEQ ID NO: 9, a LCDR1 of SEQ ID NO: 10, a LCDR2 of SEQ ID NO: 11 and a LCDR3 of SEQ ID NO: 12,
wherein the three or more 21-day cycles comprise Cycle 1, Cycle 2, and Cycle 3, and
wherein the administering comprises:
administering on day 1 of Cycle 1 the bispecific antibody at about 350 mg, pemetrexed at 500 mg/m2, and carboplatin to achieve AUC 5;
administering on day 2 of Cycle 1 the bispecific antibody at about 1050 mg;
administering on day 8 of Cycle 1 the bispecific antibody at about 1400 mg;
administering on day 15 of Cycle 1 the bispecific antibody at about 1400 mg;
administering on day 1 of Cycle 2 the bispecific antibody at about 1400 mg, pemetrexed at 500 mg/m2, and carboplatin to achieve AUC 5;
administering on day 1 of Cycle 3 the bispecific antibody at about 1750 mg, pemetrexed at 500 mg/m2, and carboplatin to achieve AUC 5.
46. The method of embodiment 43, wherein administering the bispecific antibody is by intravenous administration.
47. The method of embodiment 43, wherein administering the bispecific antibody is by subcutaneous administration.
48. The method of embodiment 43, wherein carboplatin is administered for not more than four cycles.
49. A method of treating an epidermal growth factor receptor (EGFR)-expressing or hepatocyte growth factor receptor (c-Met) expressing cancer in a subject weighing 80 kg or more, the method comprising
administering to the subject in three or more 21-day cycles a combination therapy comprising:
(a) pemetrexed and carboplatin,
(b) a bispecific antibody comprising an isolated bispecific anti-EGFR/c-Met antibody comprising a first domain comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and a second domain comprising a HCDR1 of SEQ ID NO: 7, a HCDR2 of SEQ ID NO: 8, a HCDR3 of SEQ ID NO: 9, a LCDR1 of SEQ ID NO: 10, a LCDR2 of SEQ ID NO: 11 and a LCDR3 of SEQ ID NO: 12,
wherein the three or more 21-day cycles comprise Cycle 1, Cycle 2, and Cycle 3, and
wherein the administering comprises:
administering on day 1 of Cycle 1 the bispecific antibody at about 350 mg, pemetrexed at 500 mg/m2, and carboplatin to achieve AUC 5;
administering on day 2 of Cycle 1 the bispecific antibody at about 1400 mg;
administering on day 8 of Cycle 1 the bispecific antibody at about 1750 mg;
administering on day 15 of Cycle 1 the bispecific antibody at about 1750 mg;
administering on day 1 of Cycle 2 the bispecific antibody at about 1750 mg, pemetrexed at 500 mg/m2, and carboplatin to achieve AUC 5;
administering on day 1 of Cycle 3 the bispecific antibody at about 2100 mg, pemetrexed at 500 mg/m2, and carboplatin to achieve AUC 5.
50. The method of embodiment 47, wherein administering the bispecific antibody is by intravenous administration.
51. The method of embodiment 47, wherein administering the bispecific antibody is by subcutaneous administration.
52. The method of embodiment 47, wherein carboplatin is administered for not more than four cycles.
53.

### EXAMPLES

### Example 1. Safety and Efficacy of Combination Therapy for Treating an EGFR-Expressing or c-Met Expressing Cancer in a Subject

This study is a first-in-human, open-label, multicenter Phase I to evaluate the safety, PK, and efficacy of a combination therapy (a bispecific antibody amivantamab administered on a 21-day cycle, a third generation EGFR TKI (lazertinib), and chemotherapeutic agents carboplatin and pemetrexed) in subjects with advanced NSCLC.

### 1. Study Overview: Lazertinib, Amivantamab, and Chemotherapy (LACP) Combination

Phase 1b LACP combination cohort began with lazertinib 240 mg as a starting dose administered orally once daily (QD) in combination with amivantamab, and standard doses of platinum-based chemotherapy (carboplatin and pemetrexed) in a 21-day cycle for 4 cycles followed by maintenance with lazertinib, amivantamab and pemetrexed until disease progression or unacceptable toxicities. Participants initially receive amivantamab at a dose of 1400 mg for participants weighing less than 80 kg and 1750 mg for participants weighing 80 kg or more by IV infusion once weekly (first dose will be split into 350 mg on Cycle 1 Day 1 and 1050 mg for participants weighing less than 80 kg and 1400 mg for participants weighing 80 kg or more on Cycle 1 Day 2) through Cycle 2 Day 1. Starting with Cycle 3 Day 1, participants receive amivantamab at a dose of 1750 mg for participants weighing less than 80 kg and 2100 mg for participants weighing 80 kg or more on Day 1 of each 21-day cycle.

The study may determine a LACP combination recommended Phase 2 chemotherapy dose (RP2ChD) based on available pharmacokinetic (PK) and tolerability data if no dose-limiting toxicity (DLT) is observed. After identification of the RP2ChD, the study may enroll additional participants, up to a total of 20 participants at this dose. If DLT is observed for lazertinib at 240 mg, the study may recommend reducing the dose of lazertinib sequentially to 160 mg or 80 mg per the dose de-escalation schedule. Objectives and endpoints of the study are shown in Table 1.

TABLE 1
Objectives and Endpoints of the study.

| | |
|---|---|
| Objectives | Endpoints |

Primary

| | |
|---|---|
| To identify the RP2ChD of lazertinib | Frequency of DLT |
| when combined with amivantamab and standard | |
| of care chemotherapy and to determine the | |
| tolerability of the LACP combination (Phase 1b | |
| LACP combination cohort) | |
| To characterize the safety and | Adverse events (AEs) |
| tolerability of lazertinib at the RP2ChD and | defined by the National Cancer |
| amivantamab and standard of care chemotherapy | Institute Common Terminology |
| in participants with advanced or metastatic | Criteria for Adverse Events (NCI- |
| EGFR-mutated NSCLC (Phase 1b LACP | CTCAE) Criteria Version 5.0 in |
| combination cohort) | participants treated with the LACP |
| | combination regimen |

Secondary

| | |
|---|---|
| To characterize the PK and | Serum amivantamab PK |
| immunogenicity of amivantamab in the LACP | parameters and incidence of |
| combination cohort | antibodies to amivantamab |
| To characterize the PK of lazertinib in | Plasma lazertinib PK |
| the LACP combination cohort | parameters |

### Overall Design

This is a Phase 1b open-label, multicenter study in participants with advanced EGFR-mutant NSCLC to evaluate the safety, tolerability, PK, and establish the RP2ChD regimen(s) of lazertinib in combination with amivantamab and with standard of care platinum-based chemotherapy (carboplatin and pemetrexed) (Phase 1b LACP combination cohort).

Phase 1b LACP combination cohort began with lazertinib 240 mg as a starting dose administered orally once daily (QD) in combination with amivantamab, and standard of care platinum-based chemotherapy (carboplatin and pemetrexed) in a 21-day cycle for 4 cycles followed by maintenance with lazertinib, amivantamab and pemetrexed until disease progression or unacceptable toxicities.

The study will determine a LACP combination RP2ChD based on available PK and tolerability data if no DLT is observed. After identification of the RP2ChD for the LACP combination, the study will enroll up to approximately 20 evaluable participants at this dose. If dose-limiting toxicity (DLT) is observed at 240 mg of lazertinib, the study may recommend reducing the dose of lazertinib sequentially to 160 mg or 80 mg per the dose de-escalation schedule. Up to 6 participants can be enrolled initially for 3 DLT-evaluable participants. DLT period will be 21 days or the first cycle of combination therapy. The DLT cohort will be reviewed by the study to assess the tolerability and determine the recommended dose of lazertinib in combination with amivantamab and chemotherapy.

### Phase 1b LACP Combination Cohort

Up to 6 participants are enrolled in a traditional 3+3 design, to achieve at least 3 DLT-evaluable participants. In the event that the initial dose of lazertinib is not deemed tolerable with amivantamab and chemotherapy (carboplatin-pemetrexed), then dose de-escalation may be pursued.

### Dosage and Administration of LACP Combination Cohort (Phase 1b)

Lazertinib is administered orally QD in 21-day cycles. During Cycle 1, amivantamab is administered once weekly as an iv infusion (first dose is split into 2 doses and given on Cycle 1 Day 1 and Cycle 1 Day 2) until Cycle 2 Day 1. In subsequent cycles, amivantamab is administered every 3 weeks.

On Day 1 of each cycle, the administration of chemotherapy (pemetrexed and carboplatin) and amivantamab should occur in the following order: 1) pemetrexed, 2) carboplatin, and 3) amivantamab.

The duration for carboplatin and pemetrexed infusions should follow local guidelines and regulatory labeling (eg, pemetrexed can be given over approximately 10 to 15 minutes and carboplatin can be given over approximately 30 to 60 minutes).

### Pharmacokinetic Evaluations

### Lazertinib

Plasma samples are collected for the evaluation of PK of lazertinib at the designated timepoints. Individual PK parameters are estimated, and descriptive statistics calculated for each dose level.

### Amivantamab

Serum samples are collected for the evaluation of PK of amivantamab at the designated timepoints. Individual PK parameters are estimated, and descriptive statistics calculated.

### Immunogenicity Evaluations

Serum samples are screened for antibodies binding to amivantamab and the titer of confirmed positive samples reported. Other analyses may be performed to further characterize the immunogenicity of amivantamab.

### Pharmacodynamic And Biomarker Evaluations

Blood samples are collected to undergo circulating tumor deoxyribonucleic acid (ctDNA) analysis.

Collected and archival tumor tissue samples may be evaluated for biomarkers relevant to cancer and/or analyzed to confirm ctDNA results. Levels of EGFR and cMet protein may also be assessed in tissue samples.

### Safety Evaluations

The safety of lazertinib and amivantamab is assessed by physical examinations, Eastern Cooperative Oncology Group criteria for performance status, laboratory tests, vital signs, electrocardiograms, baseline ophthalmologic examination, echocardiography or multigated acquisition, and concomitant medication usage. AEs that occur between the signing of the informed consent through 30 days following the last dose of study intervention will be recorded. The severity of AEs will be assessed using NCI-CTCAE, Version 5.0.

### Statistical Methods

The Phase 1b LACP combination cohort is conducted using a traditional 3+3 design, in which up to 6 participants are enrolled to achieve at least 3 DLT-evaluable participants at the first dose level of lazertinib (240 mg). Additional dose cohorts may be evaluated in a dose de-escalation schedule with lazertinib doses of 160 mg and then 80 mg, if needed (modified in 80 mg decrements with minimum dose of 80 mg). Once tolerability is demonstrated and a RP2ChD identified, additional participants may be enrolled (up to approximately 20 participants) to further characterize the safety, tolerability, and PK of lazertinib at the recommended dose within the 21-day dosing regimen, in combination with amivantamab and chemotherapy (RP2ChD).

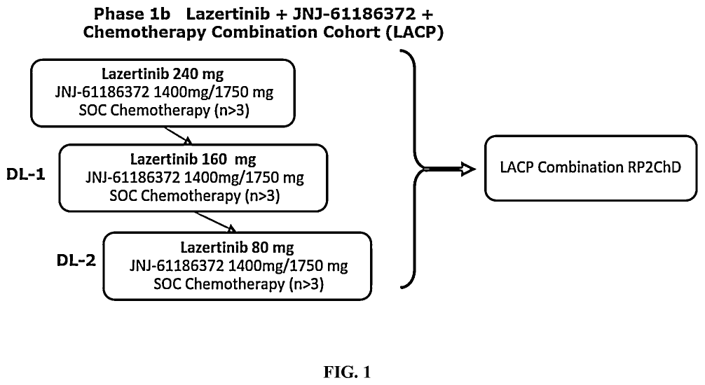

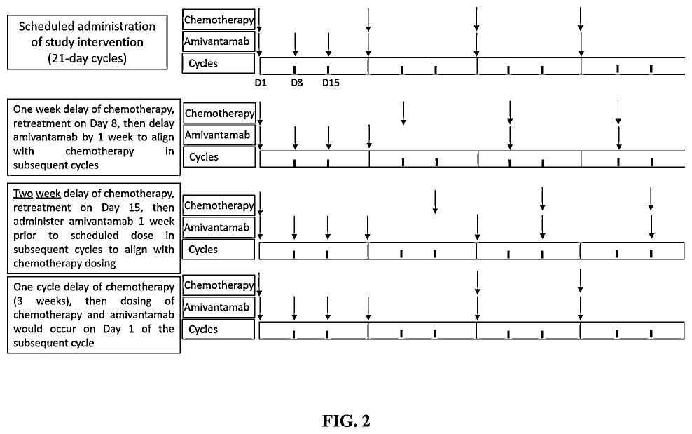

An anticipated schematic overview of the study is provided in FIG. 1.

### Schedule of Activities

Table 2 details the schedule of activities for phase 1b lazertinib, amivantamab, and chemotherapy combination (LACP) cohort.

Table 3 details the schedule of activities for pharmacokinetics and immunogenicity for Phase 1b LACP combination cohort.

TABLE 2
Schedule of Activities for Phase 1b Lazertinib, amivantamab,
and Chemotherapy Combination (LACP) Cohort

| Study Period | | | | | | | | | |
|---|---|---|---|---|---|---|---|---|---|
| | | | | | | | | | End of Treatment |
| | Treatment (21 days/cycle) | | | | | | | | Up to 30 days |
| Screening | Cycle 1 | | | | Cycle 2 | Cycle 3 | Cycle 4 | Cycle 5+ | after last dose^{v} |
| Cycle Day | | | | | | | | | |
| −28 of C1D1 | 1 | 2 | 8 | 15 | 1 | 1 | 1 | 1 | |
| Visit Window (Days) | | | | | | | | | |
| −28 to −1 | — | — | ±1 | ±1 | ±1 | ±3 | ±3 | ±3 | +7 |

STUDY PROCEDURES^{a}
SCREENING ASSESSMENTS^{b}

| | | | | | | | |
|---|---|---|---|---|---|---|---|
| Informed | X | | | | | | |
| consent^{c} | | | | | | | |
| Inclusion/exclusion | X | | | | | | |
| criteria | | | | | | | |
| Demography^{d} | X | | | | | | |
| Disease | X | | | | | | |
| characteristics^{e} | | | | | | | |
| Medical | X | | | | | | |
| history^{f} | | | | | | | |
| ECOG | X | X^{g} | X | X | X | X | X |
| performance | | | | | | | |
| status | | | | | | | |
| CT/MRI | X | | | | | | |
| imaging^{h} | | | | | | | |

STUDY INTERVENTION ADMINISTRATION

| Carboplatin | X | | | | X | X | X | |
|---|---|---|---|---|---|---|---|---|
| dosingⁱ | | | | | | | | |
| Pemetrexed | X | | | | X | X | X | X |
| dosingⁱ | | | | | | | | |
| Vitamin B12 | dose at least 7 days prior to Cycle 1 Day 1, then every 3 | | | | | | | |
| | cycles afterwards, Vitamin B12 may be administered on | | | | | | | |
| | the same day as pemetrexed | | | | | | | |
| Folic acid | Daily beginning 7 days prior to first infusion and | | | | | | | |
| | continuing until 21 days after the last dose of | | | | | | | |
| | pemetrexed | | | | | | | |
| Lazertinib | Once daily oral administration | | | | | | | |
| administration | | | | | | | | |
| amivantamab | X | X | X | X | X | X | X | X |
| administration^{j} | | | | | | | | |
| Intervention | X | X | X | X | X | X | X | X |
| compliance | | | | | | | | |

| | |
|---|---|
| Concomitant | Continuous |
| medications | |

SAFETY ASSESSMENTS^{k}

| Adverse | Continuous | | | | | | | | | |
|---|---|---|---|---|---|---|---|---|---|---|
| events | | | | | | | | | | |
| Hematology | X | X^{g} | | X | X | X | X | X | X | X |
| Serum | X | X^{g} | | X | X | X | X | X | X | X |
| chemistry | | | | | | | | | | |
| Coagulation | X | As clinically indicated | | | | | | | | |
| Serology | X | As clinically indicated | | | | | | | | |
| Urinalysis^{l} | X | As clinically indicated | | | | | | | | |
| Pregnancy | X | As clinically indicated, by urine or serum test | | | | | | | | |
| test^{m} | | | | | | | | | | |
| 12-lead | Xⁿ | X^{o} | | | | | X^{o} | As clinically | | |
| Electrocardiogram | | | | | | | | indicatedn | | |
| Echocardiography/ | X | On study assessment at 6 weeks ±1 week (relative to C1D1) then as | | | | | | | | |
| multigated | | clinically indicated thereafter | | | | | | | | |
| acquisition scan | | | | | | | | | | |
| Ophthalmologic | X | As clinically indicated | | | | | | | | |
| examination^{p} | | | | | | | | | | |
| Vital signs^{q} | X | X | X | X | X | X | X | X | X | X |
| Physical | X | X | | X | X | X | X | X | X | X |
| examination^{r} | | | | | | | | | | |

EFFICACY ASSESSMENT

| | | |
|---|---|---|
| Disease | X | X |
| assessment^{s} | | |

PHARMACOKINETICS AND IMMUNOGENICITY

| | |
|---|---|
| Pharmacokinetic | Refer to Table 3 |
| and immunogenicity | |
| blood sampling | |

BIOMARKERS

| | | | | |
|---|---|---|---|---|
| Blood sample | X | X | X | X |
| for ctDNA^{t} | | | | |
| Archival | X | | | |
| tumor tissue | | | | |
| for molecular | | | | |
| profiling (if | | | | |
| available)^{u} | | | | |

C = cycle; ctDNA = circulating tumor deoxyribonucleic acid; D = day; ECOG = Eastern Cooperative Oncology Group; EGFR = epidermal growth factor receptor; HGF = hepatocyte growth factor; min = minute(s).
^{a}Assessments on dosing days should be performed prior to administration of study intervention unless otherwise stated. Investigators can confirm that participants meet retreatment criteria prior to administration of study intervention (except for amivantamab administration on Cycle 1 Day 2).
^{b}Participants who fail to meet the inclusion and/or meet the exclusion criteria and are screen failed may be rescreened if their condition changes but can sign a new informed consent form. Rescreening can be discussed with and approved by the sponsor on a case-by-case basis.
^{c}Informed consent can be signed before any study-related procedures are performed. If an assessment was performed as part of the participant's routine clinical evaluation and not specifically for this study, it need not be repeated after signed informed consent has been obtained, provided the assessments fulfill the study requirements.
^{d}Includes age (or year of birth), gender, ethnicity, and race.
^{e}Includes tumor type and date of initial diagnosis and metastatic disease, tumor stage at initial diagnosis, histology and EGFR mutation status, prior anticancer therapies (number and type of prior regimens, best response, and duration of therapy) and date of most recent disease progression.
^{f}Includes relevant past medical diagnoses, and surgeries/procedures as well as current medical conditions with toxicity grade (including current cancer-related symptoms).
^{g}If performed within 3 days prior to the first study intervention administration, the assessment does not have to be repeated at Cycle 1 Day 1.
^{h}CT scan of the chest (including the supraclavicular region), abdomen, pelvis, and any other disease location performed with IV contrast. Participants not able to undergo CT scans with IV contrast (eg, due to allergy or renal insufficiency) may have noncontrast CT of the thorax and MRI of the abdomen and pelvis with IV contrast at baseline and during the study, if approved by the sponsor. Contraindications to the CT scan with IV contrast that develop postbaseline should be discussed with the sponsor medical monitor. MRI should be used to evaluate sites of disease that cannot be adequately imaged using CT (eg, brain). Other types of imaging, eg, bone scintigraphy, may also be conducted per the Investigator's clinical judgment and local standard of care. RECIST v1.1 guidance regarding the use of imaging techniques for determination of disease progression should also be followed. Brain MRI is required at Screening for all participants. Scans will serve as the baseline for Disease Assessment/Tumor Response.
ⁱWhen chemotherapy and amivantamab are administered on the same day, pemetrexed (and carboplatin, up to Cycle 4) should be administered before amivantamab.
^{j}Any missed scheduled doses in Cycle 1 should be discussed with medical monitor prior to redosing. In other cycles, refer to specific toxicity management guidance. Safety assessments should be aligned with dosing visits regardless of the scheduled visit window. See FIG. 2 for options to synchronize the dose of amivantamab and chemotherapy.
^{k}Results of the screening assessments can be reviewed by the Investigator prior to enrollment; on dosing days, laboratory assessments can be reviewed by the Investigator prior to administration of any study intervention. From C2D1, laboratory assessments may be performed the day before the administration of study intervention.
^{l}Perform urine microscopy if abnormal
^{m}Women of childbearing potential only. Serum or urine test required at screening and within 24 hours prior to the first study intervention administration. At other times during study, a serum or urine pregnancy test may be performed as indicated.
ⁿSingle assessment after participant has rested in a supine position for at least 5 minutes. Clinically significant abnormal results should be confirmed by triplicate assessments with repeat ECGs obtained as closely as possible in succession. Results will be recorded.
^{o}12-lead ECGs (in triplicate, approximately 2 min apart) should be performed on C1D1 prior to the dose of lazertinib and on C3D1 after EOI (within 30 minutes) of amivantamab (between 2-3 h after the administration of lazertinib).
^{p}Ophthalmic assessment, including slit lamp examination, fundoscopic examination, and eye examination (includes visual acuity and tonometry) will be performed at screening, with baseline examination results being collected, and photographs being performed to document baseline status.
^{q}Includes heart rate, blood pressure, temperature, respiratory rate, and oxygen saturation. Vital signs should be measured within 15 minutes prior to the administration of lazertinib (predose), within 30 minutes prior to each infusion of amivantamab, at approximately 30 minute intervals (+5 minutes) during each infusion of amivantamab, and at EOI (+5 minutes, End of Infusion). If pre-lazertinib vital signs are collected ≤30 minutes prior to the infusion of amivantamab, then a repeat assessment prior to the infusion of amivantamab is not required. If lazertinib is not administered, predose vital signs should be measured within 30 minutes prior to amivantamab infusion.
^{r}At all visits after the first dose of study intervention administration, participants should be questioned for skin and eye symptoms, with physical examination as appropriate, and specialty referral as indicated. On Day 1 of each cycle, weight should be obtained and directed physical examinations of involved organs and other body systems, as indicated, should be performed.
^{s}Perform CT, MRI, and other imaging/examination, if applicable, of all active disease sites at Screening, at 6 (+1) weeks (ie, no earlier than Day 42), and then every 6 (±1) weeks relative to Cycle 1 Day 1; repeat CNS imaging may be performed as clinically indicated during the Treatment Period. Results will be recorded. The same methodology should be used throughout the study. Tumor response evaluation will be assessed by the investigator according to RECIST v1.1. Results of the assessment should be available prior to the next scheduled dose. For participants who discontinue treatment prior to disease progression, radiological assessments should continue to be performed as per the protocol schedule until disease progression is documented. If a discontinued participant begins a new cancer therapy prior to progression, disease assessment should occur prior to initiation of the new therapy.
^{t}Single sample collection for exploratory ctDNA assessment prior to dosing at Screening and within 2 hours prior to dosing at Cycle 3 Day 1 and Cycle 5 Day 1, and at the End of Treatment visit 30 (+7) days after the last dose. If participant is treated beyond disease progression, an additional ctDNA sample will be collected at time of PD and prior to any systemic anticancer therapy.
^{u}Archival sample from the most recent biopsy (preferably post EGFR-TKI eg, osimertinib) should be submitted, if available. If not available, prior tumor tissue may be submitted.
^{v}End of Treatment visit occurs when all study drugs are permanently discontinued.

TABLE 3
Schedule of Activities for Pharmacokinetics and Immunogenicity
(for Phase 1b LACP combination cohort)

| | | | | | | | Immuno- |
|---|---|---|---|---|---|---|---|
| | | | | | PK^{a} | PK | genicity |
| Study Day^{x,bb} | | Sampling Time | Time Allowance | | (Lazertinib) | (amivantamab) | (amivantamab) |
| Cycle | Day 1 | Predose/Preinfusion^{y} | (0-2 | hr) | X | X | X |
| 1 | | EOI (amivantamab) ^{z} | (0-15 | min) | | X | |
| | | 2 hr post-lazertinib | +/−30 | min | X | | |
| | | administration | | | | | |
| | | 4 hr post-lazertinib | +/−1 | hr | X | | |
| | | administration | | | | | |
| | Day 2 | Predose/Preinfusion^{y} | (0-20 | min) | | X | |
| | | EOI (amivantamab) ^{z} | (0-15 | min) | | X | |
| | Day 8 | Predose/Preinfusion^{y} | (0-20 | min) | X | X | |
| | | EOI (amivantamab) | (0-15 | min) | | X | |
| | | 2 hr post-lazertinib | +/−30 | min | | | |
| | | administration | | | | | |
| | Day 15 | Predose/Preinfusion^{y} | (0-2 | hr) | X | X | |
| | | EOI (amivantamab) ^{z} | (0-15 | min) | | X | |
| | | 2 hr post-lazertinib | +/−30 | min | X | | |
| | | administration | | | | | |
| Cycle | Day 1 | Predose/Preinfusion^{y} | (0-2 | hr) | X | X | X |
| 2 | | EOI (amivantamab) ^{z} | (0-15 | min) | | X | |
| | | 2 hr post-lazertinib | +/−30 | min | X | | |
| | | administration | | | | | |
| | | 4 hr post-lazertinib | +/−1 | hr | X | | |
| | | administration | | | | | |
| Cycle | Day 1 | Predose/Preinfusion^{y} | (0-2 | hr) | X | X | X |
| 3 | | EOI (amivantamab) ^{z} | (0-15 | min) | | X | |
| Cycle | Day 1 | Predose/Preinfusion^{y} | (0-2 | hr) | X | X | X |
| 4+^{aa} | | EOI (amivantamab) ^{z} | (0-15 | min) | | X | |

| | | | |
|---|---|---|---|
| End-of-treatment | X | X | X |

EOI = end of infusion; hr = hour(s); min = minute(s); PK = pharmacokinetics.
^{w}Lazertinib is dosed before start of infusions for chemotherapy.
^{x}Assessment should be performed on the actual day of study intervention administration, not on the originally scheduled administration day.
^{y}Predose/preinfusion samples will be taken relative to their corresponding study intervention administration. Moreover, all predose samples collection should be completed before starting administration of any study intervention.
^{z} EOI sample will be taken relative to amivantamab infusion within 15 minutes of the end of infusion of amivantamab.
^{aa}After Cycle 4, sample collected on Cycles 6, 8, 10, 12, 18, 24 and every 13 cycles thereafter until end of treatment.
^{bb}Samples should be collected based on Cycle 1 Day 1 and Day 2 schedules when amivantamab is administered as a split dose.

### 2. Study: Lazertinib, Amivantamab, and Platinum-Doublet Chemotherapy (LACP) Combination

Phase 1b LACP combination cohort

To achieve at least 3 DLT-evaluable participants, 3 to 6 participants receive lazertinib 240 mg as a starting dose administered orally QD in combination with amivantamab, and standard doses of platinum-based chemotherapy (carboplatin and pemetrexed) in a 21-day cycle for 4 cycles followed by maintenance with lazertinib, amivantamab and pemetrexed until disease progression or unacceptable toxicities. Participants initially receive amivantamab at a dose of 1400 mg for participants weighing less than 80 kg and 1750 mg for participants weighing 80 kg or more by IV infusion once weekly (1^{st} dose will be split into 350 mg on Cycle 1 Day 1 and 1050 mg on Cycle 1 Day 2 for participants weighing less than 80 kg and 1400 mg for participants weighing 80 kg or more) up through Cycle 2 Day 1). Starting with Cycle 3 Day 1, participants receive amivantamab at a dose of 1750 mg for participants weighing less than 80 kg and 2100 mg for participants weighing 80 kg or more on Day 1 of each 21-day cycle. If none of the first 3 DLT-evaluable participants experiences a DLT, the study may consider that dose as the LACP combination RP2ChD. If 1 of the first 3 DLT-evaluable participants experiences a DLT, at least 3 additional participants will be enrolled and treated at that dose level for safety. Under these circumstances, all additional participants in the cohort can complete Cycle 1 of treatment and if there is no occurrence of a DLT in any additional DLT-evaluable participants (ie, <33% of participants at the current dose level have experienced DLT). If the initial lazertinib dose of 240 mg is found to be intolerable (ie, DLTs observed in ≥33% of participants), the study may recommend exploring cohorts of reduced lazertinib dosing of 160 mg, and then 80 mg per the dose de-escalation schedule in 3 to 6 participants per cohort. The LACP combination RP2ChD will be no higher than the dose level with <33% of participants experiencing a DLT. After identification of the lazertinib RP2ChD, the study may recommend additional enrollment, up to approximately 20 participants at this dose.

The Phase 1b LACP combination cohort(s) is divided into 2 periods: 1) a Screening Period, in which a participant's eligibility is reviewed prior to administration of the first dose of study intervention; 2) a Treatment Period, which starts with the first dose administration and continues until End-of-treatment visit.

### Definition of Dose-Limiting Toxicity

Toxicities, defined as study intervention-related adverse events, are graded for severity according to the NCI-CTCAE, Version 5.0. Only toxicities that occur during Cycle 1, covering the time period from the start of the first lazertinib administration through Day 21 (Phase 1) are used for the purpose of defining DLT and for dose escalation decisions. For participants in the Phase 1b LACP combination cohort, the DLT period begins with the initiation of lazertinib on Cycle 1 Day 1, and end on Cycle 1 Day 21 (21-day DLT period). Dose-limiting toxicity is defined as any of the following:

### Grade ≥3 Nonhematologic Toxicity.

Exceptions are as follows:
   Grade 3 nausea, vomiting, or diarrhea responsive to symptomatic treatment
   Grade 3 fatigue in a participant with Grade 1 or 2 fatigue at baseline
   Transient Grade 3 laboratory abnormalities that are not felt to be clinically significant by the investigator
   Rash that improves to Grade <3 with standard management within 5 days
      Liver enzyme elevation meeting these criteria: alanine aminotransferase (ALT)≥3× upper limit of normal (ULN) (if baseline was normal; ≥3.0× baseline if baseline was abnormal for LACP cohort) and total bilirubin ≥2×ULN (if baseline was normal; ≥2.0× baseline if baseline was abnormal for LACP cohort) (>35% direct bilirubin) with no evidence of alkaline phosphatase (ALP) elevation or ALT≥3×ULN (if baseline was normal; ≥3.0× baseline if baseline was abnormal for LACP cohort) and international normalized ratio (INR)>1.5 with no ALP elevation. Exception to the bilirubin elevation is made if the participant has Gilbert's disease and the elevated bilirubin is predominantly unconjugated.
The event, if confirmed via repeat laboratory assessment, should also be reported as a serious adverse event (SAE).

For the LACP combination cohort, participant receives <80% of the planned total dose of lazertinib during Cycle 1 (ie, <4,032 mg over 21 days) due to toxicity.

### Dose De-Escalation for LACP Combination Cohort

In the Phase 1b LACP combination cohort, up to 6 participants are enrolled to achieve at least 3 DLT-evaluable participants. In the event that the initial dose of lazertinib is not deemed tolerable with amivantamab and chemotherapy (carboplatin-pemetrexed), then dose de-escalation may be pursued, according to the planned strategy outlined in Table 4 and FIG. 1, and safety in the already dosed participants, after study review and agreement. PK profiles will be reviewed by the study when they become available.

TABLE 4
Lazertinib Dose De-escalation Schedule

| | [symbol] tinib Dose to be administered with |
|---|---|
| | amivantamab + Chemotherap [symbol] |
| Initial Dose | 240 mg |
| DL-1 | 160 mg |
| DL-2 | 80 mg |

[symbol]

indicates data missing or illegible when filed

### Starting Dose for the Phase 1b LACP Combination Cohort

The study includes a starting dose of amivantamab at 1400 mg for participants <80 kg and 1750 mg≥80 kg on Cycle 1 Day 1/Day 2, Day 8, and Day 15, and Cycle 2 Day 1 and 240 mg of lazertinib in combination with standard chemotherapy with a possible dose de-escalation scheme. In the event that the starting dose of lazertinib is not deemed tolerable with amivantamab and chemotherapy then dose de-escalation will be pursued, according to the planned strategy outlined in Table 4, based upon the safety in the dosed participants.

### Study Population

In the Phase 1b LACP combination cohort, participants are patients with advanced or metastatic EGFR-mutated NSCLC who have progressed on or after an EGFR-TKI as the most recent line of treatment with a maximum of 3 prior lines of therapy in the metastatic setting.

### PK and Immunogenicity Assessments

Plasma samples are collected for the evaluation of PK of Lazertinib. Refer to PK Table 3 for Phase 1b LACP combination cohorts (Table 3).

Serum samples are collected for the evaluation of PK and immunogenicity of amivantamab. Refer to PK Table 3 for Phase 1b LACP combination cohorts (Table 3).

### End of Study Definition

The end of study is considered as the last scheduled study assessment shown in the Schedule of Activities (Error! Reference source not found. and Table 3) for the last participant in the study, or the termination of the study by the sponsor, whichever comes first. The final data from the study site will be sent to the sponsor (or designee) after completion of the final participant assessment at that study site, in the time frame specified in the Clinical Trial Agreement.

### Study Completion Definition

A participant is considered to have completed the study if the participant has died before the end of the study or has not been lost to follow-up or withdrawn consent by the end of the study.

### 3. Study Population

Screening for eligible participants is performed within 28 days before administration of the study intervention.

### Inclusion Criteria

Each participant satisfies all of the following criteria to be enrolled in the study:
Participant will be ≥18 years of age or the legal age of consent in the jurisdiction in which the study is taking place
For the Phase 1b LACP combination cohort: Histologically or cytologically confirmed advanced or metastatic EGFR-mutated NSCLC who have progressed on or after an EGFR-TKI as the most recent line of treatment with a maximum of 3 prior lines of therapy in the metastatic setting allowed.
Evaluable disease: Participant will have measurable disease according to RECIST v1.1. Measurable lesions should not have been biopsied during screening, but if only 1 non-irradiated measurable lesion exists, it may undergo a biopsy and be acceptable as a target lesion, provided the baseline tumor assessment scans are performed at least 7 days after the biopsy.
Participant will have ECOG performance status grade of 0 or 1
Organ and bone marrow function as shown in Table 5:

TABLE 5

| | |
|---|---|
| LABORATORY | |
| PARAMETER | VALUE |

Hematology

| | |
|---|---|
| Hemoglobin | ≥9 g/dL (≥10 g/dL for LACP combination |
| | cohort) |
| | ≥1.5 × 10⁹/L |
| Platelets | ≥75 × 10⁹/L (≥100 × 10⁹/L for LACP |
| | combination cohort) |

Hepatic and Renal

| | |
|---|---|
| AST and ALT | ≤3 × ULN |
| Total bilirubin | ≤1.5 × ULN; participants with Gilbert's |
| | syndrome can enroll if conjugated |
| | bilirubin is within normal limits |
| Serum creatinine or | Creatinine clearance >50 mL/min |
| creatinine clearance^{a} | |

ALT = alanine aminotransferase; ANC = absolute neutrophil count; AST = aspartate aminotransferase; ULN = upper limit of normal; min = minute(s).
^{a}creatinine clearance will be calculated by Cockroft Gault formula and should be >50 mL/min.

Cockcroft-Gault Formula for Estimated Creatinine Clearance for Adults is calculated according to Equation 1:

$$= \frac{( 140 - Age ) \times Mass ( Kilograms ) \times [ 0.85 if female ]}{72 \times Serum Creatinine ( in mg / dL )}$$

or according to Equation 2:

$$e C r C l = \frac{( 140 - Age ) \times Mass ( Kilograms ) \times Constant}{Serum Creatinine ( in μ mol / L )}$$

where Constant=1.23 for men and 1.04 for women.
Participants meet the above laboratory criteria without having a history of red blood cell transfusion, platelet transfusion, or granulocyte-colony stimulating factor support within 7 days prior to the date of the test.
Prior to enrollment, a woman is either not of childbearing potential OR of childbearing potential and practicing a highly effective, user-independent method of contraception and one additional method and agrees to remain on highly effective method while receiving study intervention and until 6 months after last dose
A woman of childbearing potential:
   Must have a negative serum or urine β human chorionic gonadotropin at screening.
   Must agree not to breast-feed during the study and for 6 months after the last dose of study intervention. (Enrollment is not allowed even if a woman who is breast-feeding stops breast-feeding).
   Must agree not to donate eggs (ova, oocytes) for the purposes of assisted reproduction during the study and for 6 months after receiving the last dose of study intervention.
A man who is sexually active with a woman of childbearing potential agrees to continue contraception throughout the study including up to 6 months after the last dose of study intervention.
Willing and able to adhere to the prohibitions and restrictions (Prohibited Medications and Therapies for Lazertinib) specified in this protocol.
Each participant signs an informed consent form (ICF) indicating that he or she understands the purpose of and procedures required for the study is willing to participate in the study.

### Exclusion Criteria

Any potential participant who meets any of the following criteria is excluded from participating in the study:
1. Participant has an uncontrolled illness, including but not limited to:
   Uncontrolled diabetes
   Ongoing or active infection (includes infection requiring treatment with antimicrobial therapy [participants will be required to complete antibiotics 1 week prior to starting study treatment] or diagnosed or suspected viral infection).
   Active bleeding diathesis
   Impaired oxygenation requiring continuous oxygen supplementation
   Refractory nausea and vomiting, chronic gastrointestinal diseases, inability to swallow the formulated product, or previous significant bowel resection that would preclude adequate absorption of study treatment
   Psychiatric illness or any other circumstances (including social circumstances) that would limit compliance with study requirements
   Any ophthalmologic condition that is either clinically unstable or requires treatment
2. Prior treatment with antiPD-1 or antiPD-L1 antibody within 6 weeks of planned first dose of study intervention.
3. Prior chemotherapy, targeted cancer therapy, immunotherapy (refer to Exclusion Criterion #2 for antiPD-1 or antiPD-L1 therapy), or treatment with an investigational anticancer agent within 2 weeks or 4 half-lives whichever is longer, before the first administration of study intervention(s). For agents with long half-lives, the maximum required time since last dose is 4 weeks. Note: Localized radiotherapy for palliative purposes should be completed at least 7 days prior to the first administration of study intervention(s).
4. Untreated brain or other CNS metastases, whether symptomatic or asymptomatic. Participants who have completed definitive therapy, are not on steroids, and have a stable clinical status for at least 2 weeks prior to study treatment may be eligible. If brain metastases are diagnosed on Screening imaging, the participant may be enrolled, or rescreened for eligibility, after definitive treatment if above criteria are met.
5. Any toxicities from prior anticancer therapy should have resolved to CTCAE version 5.0 Grade 1 or baseline level (except for alopecia [any grade], Grade ≤2 peripheral neuropathy, and Grade ≤2 hypothyroidism stable on hormone replacement therapy).
6. History of clinically significant cardiovascular disease including, but not limited to:
   Newly diagnosed deep vein thrombosis or pulmonary embolism within 4 weeks prior to the first dose of study intervention. Clinically nonsignificant thrombosis, such as nonobstructive catheter-associated clots, are not exclusionary. Participant with a medical history of deep vein thrombosis or pulmonary embolism should be clinically stable on anticoagulant therapy for a minimum of 4 weeks prior to the first dose of study intervention.
   Any of the following within 24 weeks prior to the first dose of study intervention: myocardial infarction, unstable angina, stroke, transient ischemic attack, coronary/peripheral artery bypass graft, or any acute coronary syndrome.
   Prolonged corrected QT interval by Fredericia (QTcF) interval >480 msec or clinically significant cardiac arrhythmia or electrophysiologic disease (eg, placement of implantable cardioverter defibrillator or atrial fibrillation with uncontrolled rate or abnormalities in conduction or morphology of ECG [eg, complete left bundle branch block, third- or second-degree heart block, PR interval >250 msec]). Note: Participants with cardiac pacemakers who are clinically stable are eligible.
   Any factors that increase the risk of corrected QT interval (QTc) prolongation or risk of arrhythmic events such as hypokalemia, congenital long QT syndrome, family history of long QT syndrome, or unexplained sudden death under 40 years of age in first degree relatives or any concomitant medications known to prolong QT interval or induce Torsades de Pointes.
   Uncontrolled (persistent) hypertension: systolic blood pressure >180 mm Hg; diastolic blood pressure >100 mm Hg
   Congestive heart failure defined as New York Heart Association Class III-IV or hospitalization for congestive heart failure (any New York Heart Association Class) within 6 months prior to the first dose of study intervention.
   Pericarditis/clinically significant pericardial effusion.
   Myocarditis.
   Phase 1b lazertinib+JNJ-327 expansion and LACP combination cohort: Baseline LVEF below the lower limit of normal (LLN), as assessed by screening echocardiogram or multigated acquisition (MUGA) scan.
7. Taken any disallowed therapies, Concomitant Therapy before the planned first dose of study intervention.
8. Allergies, hypersensitivity, or intolerance to lazertinib or amivantamab or their excipients.
   For the LACP combination cohort: participant has a contraindication for the use of carboplatin or pemetrexed. Participant has a history of hypersensitivity to, or cannot take, vitamin B12 or folic acid.
9. Received an investigational intervention (including investigational vaccines, but not including anticancer therapy [refer to Exclusion Criterion #2]) or used an invasive investigational medical device within 6 weeks before the planned first dose of study intervention.
10. Had, or is scheduled to have, any of the following:
   An invasive operative procedure with entry into a body cavity or major surgery (eg, requiring general anesthesia), excluding placement of vascular access or percutaneous tumor biopsy, within 4 weeks or without complete recovery before Cycle 1 Day 1. Thoracentesis, if needed and/or clinically indicated, and percutaneous biopsy for tumor tissue sample, if needed, may be done less than 4 weeks prior to Cycle 1 Day 1, as long as the participant has adequately recovered from the procedure prior to the first dose of study intervention in the clinical judgement of the investigator.
   Significant traumatic injury within 3 weeks before the start of Cycle 1 Day 1 (all wounds should be fully healed prior to Day 1).
   Any medical condition that requires intact wound healing capacity and is expected to endanger participant safety if wound healing capacity would be severely reduced during administration of the study intervention.
   Expected major surgery while the investigational agent is being administered or within 6 months after the last dose of study intervention.
11. Investigative site personnel directly affiliated with this study.
12. Participant has at Screening:
   Positive hepatitis B (hepatitis B virus [HBV]) surface antigen (HBsAg)
   Note: Participants with a prior history of HBV demonstrated by positive hepatitis B core antibody are eligible if they have at Screening 1) a negative HBsAg and 2) an HBV DNA (viral load) below the lower limit of quantification, per local testing. Participants with a positive HBsAg due to recent vaccination are eligible if HBV DNA (viral load) is below the lower limit of quantification, per local testing.
   Positive hepatitis C (hepatitis C virus [HCV]) antibody (anti-HCV)
   Note: Participants positive for HCV antibody are eligible only if polymerase chain reaction is negative for HCV ribonucleic acid (RNA).
   Other clinically active infectious liver disease.
13. Human immunodeficiency virus antibody positive, or tests positive for human immunodeficiency virus at screening.
14. Serious underlying medical or psychiatric condition (eg, alcohol or drug abuse), dementia or altered mental status or any issue that would impair the ability of the participant to receive or tolerate the planned treatment at the study site, to understand informed consent or that in the opinion of the investigator would contraindicate the participant's participation in the study or confound the results of the study.
15. Participant has an active or past medical history of ILD/pneumonitis, including drug-induced or radiation ILD/pneumonitis.
16. Any condition for which, in the opinion of the investigator, participation would not be in the best interest of the participant (eg, compromise the well-being) or that could prevent, limit, or confound the protocol-specified assessments.
17. Has leptomeningeal metastatic disease, whether currently active or prior history thereof.
18. Participant has uncontrolled tumor-related pain (symptomatic lesions amenable to palliative radiotherapy [eg, bone metastases or metastases causing nerve impingement] should be treated prior to Screening).

All study enrollment criteria should have been met at screening. If a participant's clinical status changes (including any available laboratory results or receipt of additional medical records) after screening but before the first dose of study intervention is given such that the participant no longer meets all eligibility criteria, supportive treatment may be administered, if necessary, so that eligibility criteria can be met and laboratory test(s) may be repeated once, to determine if the participant qualifies for the study. If enrollment criteria are not met after further evaluation, then the participant should be excluded from participation in the study.

### Prohibited Medications and Therapies for Lazertinib

Lazertinib is an investigational drug. Lazertinib is considered unlikely to cause clinically significant drug interactions through induction or inhibition of cytochrome P450 (CYP) enzyme activity. Strong inducers of CYP3A4 may reduce the exposure of lazertinib when concomitantly administered.

All patients receiving lazertinib should avoid medications and herbal supplements that are known to have strong inducer effects on CYP3A4. Strong inducers of CYP3A4, including those listed below (Table 6), should be identified during screening and discontinued for an appropriate period before starting study treatment regimens that include lazertinib.

The potential for CYP 3A4/5 inhibition on lazertinib exposure is thought to be low at clinically relevant concentrations. To ensure patient safety, use of medications known to be strong inhibitors of CYP3A4 and substrates of CYP3A4/5 is restricted and should be avoided, when possible, or used with caution (Table 7).

TABLE 6
Listing of Prohibited Medications and Therapies for
Lazertinib That are Strong Inducers of CYP3A4/5
Medications Inducing CYP3A4/5

| | Withdrawal Period |
|---|---|
| | Prior To Lazertinib |
| Contraindicated drugs | Start |
| phenytoin, rifampicin, St. John's Wort, | 3 weeks |
| carbamazepine, primidone, griseofulvin, | |
| barbiturates, troglitazone, pioglitazone, | |
| oxcarbazepine, nevirapine, efavirenz, rifabutin | |
| Phenobarbitone | 5 weeks |

TABLE 7
Listing of Restricted Medications and Therapies That
Are Strong Inhibitors and Substrates of CYP3A4/5

| | |
|---|---|
| | Withdrawal |
| | Period Prior |
| | To Lazertinib |
| Contraindicated Drugs | Start |

Medications Inhibiting CYP3A4/5

| | | |
|---|---|---|
| ketoconazole, itraconazole, indinavir, saquinovir, | 1 | week |
| nelfinavir, atazanavir, amprenavir, fosamprenavir, | | |
| troleandomycin, telithromycin, fluconazole, | | |
| nefazodone, cimetidine, aprepitant, miconazole, | | |
| fluvoxamine | | |
| Amiodarone | 27 | weeks |
| erythromycin, clarithromycin, verapamil, ritonavir, | 2 | weeks |
| diltiazem | | |

Medications that are Substrates of CYP3A4

| | | |
|---|---|---|
| Abemaciclib, ABT-384, alfentanil, almorexant, alpha- | 1 | day |

dihydroergocryptine, atazanavir, avanafil,
avapritinib, brecanavir, buspirone, casopitant,
conivaptan, darifenacin, darunavir, dronedarone,
ebastine, elvitegravir, everolimus, ibrutinib,
ivacaftor, lomitapide, lopinavir, lovastatin,
maraviroc, midazolam, midostaurin, naloxegol,
nisoldipine, paritaprevir, saquinavir, simeprevir,
simvastatin, sirolimus, tacrolimus, tilidine,
tipranavir, triazolam, vardenafil, voclosporin,
zanubrutinib

This listing is not intended to be exhaustive, and a similar restriction will apply to other agents known to induce or inhibit CYP3A4/5 activity, or that are substrates of CYP3A4/5.

### 4. Dosage and Administration for the Phase 1b LACP Combination Cohort

Participants receive lazertinib in combination with amivantamab, carboplatin and pemetrexed chemotherapy in a 21-day cycle. Treatment with study medications continue until disease progression, unacceptable toxicity, or withdrawal of consent. In this cohort:

Lazertinib is dosed orally QD and is the first therapy to be administered on Day 1 of each cycle in the combination regimen.

Carboplatin and pemetrexed are administered, per standard of care, according to local guidance and regulatory labeling on Day 1 of each cycle for the first 4 cycles, followed by maintenance pemetrexed starting with Cycle 5.

Amivantamab is administered weekly within Cycle 1 with the first dose split (Day 1/Day 2, Day 8, and Day 15) and then Day 1 of every cycle thereafter. Infusion of amivantamab occurs after the completion of chemotherapy on Day 1 of each cycle (Table 8).

The sequence of administration for lazertinib, pemetrexed, carboplatin, and amivantamab is outlined in Table 9.

TABLE 8
Schedule and sequence of Dosing for Lazertinib,
amivantamab, and Combination Chemotherapy

| Agents/ | Cycle 1 | | | | Cycle 2 | Cycle 3+ |
|---|---|---|---|---|---|---|
| Drugs^{a} | Day 1 | Day 2 | Day 8 | Day 15 | Day 1 | Day 1 |
| Lazertinib | To be administered daily starting on C1D1 | | | | | |
| Pemetrexed | X | | | | X | X |
| Carboplatin^{b} | X | | | | X | X |
| amivan- | X | X | X | X | X | X |
| tamab^{c} | | | | | | |

^{a}Agents/drugs arranged sequentially in the desired order of administration
^{b}Carboplatin to stop after 4 cycles
^{c}amivantamab infusion may be delayed to Cycle 1 Days 2 and 3, if delays in pemetrexed and carboplatin infusions cause delay in amivantamab initiation.

TABLE 9
Initial Dosing for lazertinib with amivantamab
and Combination Chemotherapy

| Study Visit | | |
|---|---|---|
| Study Intervention | Study Intervention Dosage | |
| Cycle 1 (D 1-21) | | |
| Lazertinib | 240 | mg |
| Cycle 1 Day 1 | | |
| Pemetrexed | 500 | mg/m² |
| Carboplatin | AUC 5 (max 750 mg) | |
| amivantamab | 350 | mg |
| Cycle 1 Day 2 | | |
| amivantamab | 1050 mg | (<80 kg) |
| | 1400 mg | (≥80 kg) |
| Cycle 1 Day 8 and Day 15 | | |
| amivantamab | 1400 mg | (<80 kg) |
| | 1750 mg | (≥80 kg) |
| Cycle 2(D 1-21) | | |
| Lazertinib | 240 | mg |
| Cycle 2 Day 1 | | |
| Pemetrexed | 500 | mg/m² |
| Carboplatin | AUC 5 (max 750 mg) | |
| amivantamab | 1400 mg | (<80 kg) |
| | 1750 mg | (≥80 kg) |
| Cycles 3-4 (D 1-21) | | |
| Lazertinib | 240 | mg |
| Cycles 3-4 Day 1 | | |
| Pemetrexed | 500 | mg/m² |
| Carboplatin | AUC 5 (max 750 mg) | |
| amivantamab | 1750 mg | (<80 kg) |
| | 2100 mg | (≥80 kg) |
| Cycles 5+(D 1-21) | | |
| Lazertinib | 240 | mg |
| Cycles 5+ Day 1 | | |
| Pemetrexed | 500 | mg/m² |
| amivantamab | 1750 mg | (<80 kg) |
| | 2100 mg | (≥80 kg) |

### Amivantamab

amivantamab is administered as an i.v. infusion and the details of each administration recorded (including date, start and stop times of the i.v. infusion, and volume infused).

### Chemotherapy (Carboplatin and Pemetrexed)

Carboplatin and pemetrexed are administered as IV infusions according to local guidelines and/or product labeling, and the details of each administration recorded (including date, start, and stop times of the IV infusion, and volume infused).

### Prohibited Medications and Therapies

The following concomitant medications and therapies are prohibited during the study:

Any chemotherapy, anticancer therapy, or experimental therapy other than study intervention[s].

Radiotherapy to tumor lesions being assessed for tumor response prior to radiographic progression.

Concomitant use of medications, herbal supplements and/or ingestions of foods with known potent (strong) inducer or effects on CYP3A4/5 activity. Drugs that inhibit CYP3A4/5 activity should be avoided when possible or should be used with caution. Guidance on drugs that are prohibited, require close monitoring and washout periods is given in section titled “Prohibited Medications and Therapies for Lazertinib,” see also Tables 6 and Table 7.

Use of live or attenuated vaccines is prohibited.

Concomitant administration of aminoglycosides should be avoided with carboplatin.

Caution should be exercised when administering pemetrexed concurrently with a nonsteroidal anti-inflammatory drug to a participant whose creatinine clearance is <80 mL/min (exception: low-dose aspirin once daily is permitted during the study).

Avoid using ibuprofen 2 days before, the day of, and 2 days after administration of pemetrexed, as ibuprofen increases exposure of pemetrexed in patients with creatinine clearance of 45 to 79 mL/min.

### Restricted Medications and Therapies

The following concomitant medications and therapies are restricted during the study and should be avoided, when possible, or used with caution.

Lazertinib is an inhibitor of P-glycoprotein (P-gp), multidrug resistance protein 4 (MRP4), breast cancer resistance protein (BCRP) and Organic Cation Transporter 1 (OCT1). Therefore, concomitant administration of medications, herbal supplements and/or ingestions of foods that are substrates of P-gp, MRP4, BCRP or OCT1 should be used with caution. A list of substrates of P-gp, MRP4, and BCRP is provided in Table 10.

TABLE 10
Substrates of P-glycoprotein (P-gp), Multi-drug Resistance Protein
4 (MRP4), and Breast Cancer Resistance Protein (BCRP)

| Transporter | Substrates |
|---|---|
| P-gp | Dabigatran etexilate |
| | Digoxin |
| | Fexofenadine |
| | Loperamide |
| | Auinidine |
| BCRP | Rosuvastatin |
| | Sulfasalazine |
| | Coumestrol |
| | Daidzein |
| | Dantrolene |
| MRP4 | acyclovir |
| | ritonavir |
| | adefovir |
| | tenofovir |
| | furosemide |
| | hydrochlorothiazide |
| | ceftizoxime |
| | cefazolin |
| | methotrexate |

Due to the potential for hypomagnesemia associated with EGFR inhibitors, concomitant medications that may decrease serum magnesium should be avoided if possible.

The use of concomitant medications with doublet chemotherapy should follow locally approved product labels and clinical practice guidelines.

### Phase 1b LACP Combination Cohort-Prophylaxis for Chemotherapy

Consistent with standard of care guidelines, all participants receiving pemetrexed can receive concomitant treatment with corticosteroids, folic acid, and vitamin B12 according to local regulations and consistent with the pemetrexed product label.

### Dose Modification and Management of Toxicity

Participants with any grade of drug-related toxicity should be provided symptomatic treatment when applicable. If, for any grade of drug-related toxicity, the symptoms are not tolerable as per clinical assessment by the investigator, the study intervention will be withheld until the toxicity experienced returns to Grade ≤1 or baseline (excluding EGFR-TKI-related rash). A participant for whom dosing was skipped should be assessed for resolution of toxicity.

Any dose/dosage adjustment should be overseen by medically qualified study-site personnel (preferably the principal or subinvestigator) unless an immediate safety risk appears to be present. If, however, the experienced toxicity is felt to be attributable to either amivantamab or lazertinib, then the dose of the responsible agent should be preferentially reduced.

### Dose Modification Guidance

For participants receiving amivantamab and lazertinib in combination with chemotherapy, decisions regarding dose modification of these agents should be guided by the observed toxicity, the safety profile of each drug and the likelihood of causality to each agent. For toxicities associated with EGFR inhibition that require treatment modification, and which can be attributed to either drug, modification should be instituted for lazertinib prior to modification of amivantamab dosing. Dose modification of lazertinib administered within LACP combination are shown in Error! Reference source not found.

TABLE 11
Guidance for Lazertinib Stepwise Dose Reduction.

| Dose Level (DL) | Lazertinib (any cycle) |
|---|---|
| 0 (starting dose) | 240 mg |
| −1 | 160 mg |
| −2 | 80 mg |
| −3 | Discontinue |

Dose modification of amivantamab are shown in Error! Reference source not found.

TABLE 12
Guidance for amivantamab Stepwise Dose Reduction.

| | amivantamab (up to Cycle | |
|---|---|---|
| Dose Level | 2 Day 1) | amivantamab (Cycle 3+) |
| 0 (starting | 1,400 mg (1,750 mg if | 1,750 mg (2,100 mg if |
| dose) | body weight ≥80 kg) | body weight ≥80 kg) |
| −1 | 1,050 mg (1,400 mg if | 1,400 mg (1,750 mg if |
| | body weight ≥80 kg) | body weight ≥80 kg) |
| −2 | 700 mg (1,050 mg if | 1,050 mg (1,400 mg if |
| | body weight ≥80 kg) | body weight ≥80 kg) |
| −3 | Discontinue | Discontinue |

For chemotherapy dose modification, final treatment decisions should depend on clinical judgement, based on local regulations and labeling. For participants receiving amivantamab and lazertinib in combination with chemotherapy, decisions on study drug dose modification should be guided by the safety profile of each drug and the likelihood of causality.

Dose modifications of chemotherapy will be based on the maximum toxicity experienced during a cycle. Treatment should be delayed until the toxicity resolves to Grade ≤1 or the baseline status of the participant.

Participants may have a maximum of 2 dose modifications to each treatment throughout the course of the study for toxicities before the agent should be discontinued. Dose reduction should be based upon the most severe toxicity if multiple toxicities are experienced concurrently.

Treatment Delay and Dosing Synchronization of Amivantamab with Chemotherapy (Phase 1b LACP Combination Cohort)

Participants receiving combination chemotherapy meet retreatment criteria for chemotherapy per local regulations and guidelines, and amivantamab prior to redosing with respective agents.

### 6. Study Assessments and Procedures

### Overview

The Schedule of Activities (Error! Reference source not found. and Table 3) summarizes the frequency and timing of screening, efficacy, PK, immunogenicity, PD, biomarker, safety, and other measurements applicable to this study.

### Screening Period

The Screening Period starts at the time of the signing of the ICF and ends at the start of the first administration of study intervention. The Screening Period includes items listed in the Schedule of Activities Error! Reference source not found., including a review of medical history to determine eligibility for the study, signing of the informed consent, and the completion of all assessments required to verify eligibility before the initiation of therapy.

Eligibility criteria are reviewed, and a complete clinical evaluation performed. Treatment Period

The Treatment Period begins on Day 1 of Cycle 1 with the administration of the study intervention and continues until the completion of the End-of-treatment Visit. The frequency of study site visits and details of the procedures performed are outlined in the Schedule of Activities, Error! Reference source not found. and Table 3. The latest measurements taken on Day 1 Cycle 1 before administration of study intervention or at screening are defined as baseline values. An End-of-treatment Visit is scheduled up to 30 days (+7) after the last dose of study intervention for all participants, including those discontinuing study interventions for any reason, except for lost to follow-up, death, or withdrawal of consent for study participation.

Participants are evaluated throughout the Treatment Period for possible toxicities and adverse events. Adverse event information will be graded using the NCI-CTCAE, Version 5.0. All Grade 3 or Grade 4 adverse events considered related to study intervention and adverse events leading to discontinuation can be followed until recovery to baseline or Grade ≤1 or until deemed irreversible. The unresolved aforementioned events will be followed for a maximum of 6 months from the last dose of study intervention.

End-of-treatment Visit is conducted up to 30 days (+7) after the last dose of study intervention and should be completed before starting any subsequent anticancer treatment.

### Efficacy Assessments

Disease assessments are performed as scheduled regardless of any dose modifications, according to the Schedule of Activities (Error! Reference source not found.). Assessment of responses are performed according to RECIST, Version 1.1 criteria. RECIST assessments at baseline should be representative of all areas involved with metastases. At any time disease progression is clinically suspected, tumor assessments should be performed. Irradiated or partially excised lesions are generally considered not measurable at baseline and are followed as nontarget lesions, except for a lesion that has progressed following local radiation or surgery, provided the investigator and sponsor's medical monitor agree it is measurable and will not confound the efficacy evaluation. Additionally, a lesion that was biopsied during screening should only be assessed as a target lesion if a post-biopsy CT confirms that it still meets measurability criteria and is amenable to accurate and reproducible measurement. Tumor response will be reported. The following response criteria (according to RECIST, Version 1.1) are acceptable: complete response, partial response, stable disease, progressive disease, unevaluable.

A response of partial response or complete response should be confirmed by repeat assessments ≥4 weeks from the initial observation. For a response to qualify as stable disease, follow-up measurements should have met the stable disease criteria at least once at a minimum interval not less than 6 weeks after the first dose of study intervention.

If symptomatic deterioration (on the basis of global deterioration of health status) is recorded as the basis for determining disease progression, then the decision and the date should be recorded and the clinical findings used to make the determination should be recorded. Every effort should be made to document radiographic progression even after discontinuation of treatment for symptomatic deterioration, but prior to subsequent therapy, if possible.

### Treatment After Initial Disease Progression

Due to likely heterogeneity of the disease, a study intervention may continue beyond tumor progression determined on the basis of the RECIST, Version 1.1 criteria.

### Safety Assessments

Adverse events will be reported and followed by the investigator.

Any clinically relevant changes occurring during the study will be recorded.

Any clinically significant abnormalities persisting at the end of the study/early withdrawal will be followed by the investigator until resolution or until a clinically stable condition is reached.

The study includes the following evaluations of safety and tolerability according to the timepoints provided in the Schedule of Activities (Error! Reference source not found.). Physical Examinations

The screening physical examination includes, at a minimum, participant's height, weight, general appearance, examination of the skin, ears, nose, throat, lungs, heart, abdomen, extremities, musculoskeletal system, lymphatic system, and nervous system. Participants are questioned for skin and eye symptoms at all visits, with physical examinations conducted as appropriate, and specialty referral as indicated. On Day 1 of each cycle, a directed physical examination of involved organs and other body systems, as indicated, is performed and participant weight obtained.

### Vital Signs

Vital sign measurements are obtained as indicated in the Schedule of Activities (Error! Reference source not found.) and include assessment of the following:

Temperature, pulse/heart rate, respiratory rate, SpO₂ (using pulse oximeter), and blood pressure are assessed.

Blood pressure and pulse/heart rate measurements are assessed in a seated position, with a completely automated device. Manual techniques are used only if an automated device is not available.

Blood pressure and pulse/heart rate measurements should be preceded by at least 5 minutes of rest in a quiet setting without distractions (eg, television, cell phones). Electrocardiograms

Single ECG assessment after the participant has rested in a supine position for at least 5 minutes are performed locally for all participants according to the Schedule of Activities (Error! Reference source not found.) and include measurement of heart rate, QRS axis, and intervals for PR, uncorrected QT interval (QT), QRS, RR, and QTcF.

QTcF is calculated using the Fridericia's formula: QTcF=QT/(RR)\hat{ }0.33.

During the collection of ECGs, participants should be in a quiet setting without distractions (eg, television, cell phones). Participants should rest in a supine position for at least 5 minutes before ECG collection and should refrain from talking or moving arms or legs. If blood sampling or vital sign measurement is scheduled for the same timepoint as ECG recording, it is recommended that the procedures be performed in the following order: ECG(s), vital signs, and blood draw.

The investigator reviews the printout, including ECG morphology, for immediate management. Clinically significant abnormal results are confirmed by triplicate assessments with repeat ECGs obtained as closely as possible in succession. The results are recorded. Abnormalities noted at screening are to be included in the medical history.

At each time point at which triplicate ECGs are required, 3 individual ECG tracings should be obtained as closely as possible in succession (approximately 2 minutes apart).

### Clinical Safety Laboratory Assessments

Blood samples for serum chemistry, hematology, and coagulation and a urine sample for urinalysis are collected at times indicated in the Schedule of Activities (Error! Reference source not found.).

Blood samples for serum chemistry and hematology are collected at each visit prior to study intervention administration. Screening laboratory results are evaluated before the participant is enrolled in the study. Laboratory values are reviewed prior to each study intervention administration.

### ECOG Performance Status

ECOG performance status score are determined at prespecified timepoints listed in the Schedule of Activities, Error! Reference source not found.

### Ophthalmologic Examination

Ophthalmic assessment, including slit lamp examination, fundoscopic examination, and eye examination (includes visual acuity and tonometry) are performed at screening. Appropriate postbaseline ophthalmic assessments should be repeated, if clinically indicated, with any clinically significant findings, including those confirmed by the ophthalmologist, being reported as an AE. Postbaseline examination photographs (if possible) should be performed to record any clinically significant findings.

### Echocardiography or Multigated Acquisition

Echocardiography or MUGA scans (if echocardiography is not available) should be performed as provided in Schedule of Activities (Error! Reference source not found.).

### Adverse Events and Serious Adverse Events

Adverse events are reported by the participant (or, when appropriate, by a caregiver, surrogate, or the participant's legally acceptable representative) for the duration of the study.

Adverse events are recorded from the time a signed and dated informed consent is obtained until 30 days after the last dose of study intervention or until the start of subsequent anticancer therapy, if earlier. Adverse events occurring after 30 days following the last dose of study intervention should also be reported, if considered related to study intervention. Adverse events will be followed by the investigator and graded according to the NCI-CTCAE, Version 5.0.

Any clinically relevant changes occurring during the study will be recorded, including abnormal laboratory data.

### Disease-Related Events and Disease-Related Outcomes not Qualifying as Adverse Events or Serious Adverse Events

All events that meet the definition of an SAE are reported as SAEs, regardless of whether they are protocol-specific assessments.

Disease progression should not be recorded as an adverse event or serious adverse event term; instead, signs and symptoms of clinical sequelae resulting from disease progression/lack of efficacy will be reported if they fulfil the serious adverse event definition (refer to Adverse Event Definitions and Classifications below).

### Adverse Event Definitions and Classifications

### Adverse Event

An adverse event is any untoward medical occurrence in a clinical study participant administered a medicinal (investigational or noninvestigational) product. An adverse event does not necessarily have a causal relationship with the intervention. An adverse event can therefore be any unfavorable and unintended sign (including an abnormal finding), symptom, or disease temporally associated with the use of a medicinal (investigational or noninvestigational) product, whether or not related to that medicinal (investigational or noninvestigational) product. (Definition per International Council for Harmonisation [ICH]).

This includes any occurrence that is new in onset or aggravated in severity or frequency from the baseline condition, or abnormal results of diagnostic procedures, including laboratory test abnormalities.

### Serious Adverse Event

A serious adverse event based on ICH and EU Guidelines on Pharmacovigilance for Medicinal Products for Human Use is any untoward medical occurrence that at any dose:
Results in death.
Is life-threatening
   (The participant was at risk of death at the time of the event. It does not refer to an event that hypothetically might have caused death if it were more severe.)
Requires inpatient hospitalization or prolongation of existing hospitalization
Results in persistent or significant disability/incapacity

Is a congenital anomaly/birth defect.

Is a suspected transmission of any infectious agent via a medicinal product Is Medically Important.

If a serious and unexpected adverse event occurs for which there is evidence suggesting a causal relationship between the study intervention and the event (eg, death from anaphylaxis), the event will be reported as a serious and unexpected suspected adverse reaction even if it is a component of the study endpoint (eg, all-cause mortality).

### Unlisted (Unexpected) Adverse Event/Reference Safety Information

An adverse event is considered unlisted if the nature or severity is not consistent with the applicable product reference safety information. For lazertinib and amivantamab, the expectedness of an adverse event will be determined by whether or not it is listed in the Investigator's Brochure.

Adverse Event Associated with the Use of the Intervention

An adverse event is considered associated with the use of the intervention if the attribution is possible, probable, or very likely by the definitions listed below (see Attribution Definitions below).

### Attribution Definitions

### Not Related

An adverse event that is not related to the use of the intervention.

### Doubtful

An adverse event for which an alternative explanation is more likely, eg, concomitant treatment(s), concomitant disease(s), or the relationship in time suggests that a causal relationship is unlikely.

### Possible

An adverse event that might be due to the use of the intervention. An alternative explanation, eg, concomitant treatment(s), concomitant disease(s), is inconclusive. The relationship in time is reasonable; therefore, the causal relationship cannot be excluded.

### Probable

An adverse event that might be due to the use of the intervention. The relationship in time is suggestive (eg, confirmed by dechallenge). An alternative explanation is less likely, eg, concomitant treatment(s), concomitant disease(s).

### Very Likely

An adverse event that is listed as a possible adverse reaction and cannot be reasonably explained by an alternative explanation, eg, concomitant treatment(s), concomitant disease(s). The relationship in time is very suggestive (eg, it is confirmed by dechallenge and rechallenge).

### Severity Criteria

An assessment of severity grade will be made by the investigator according to the National Cancer Institute-Common Terminology Criteria for Adverse Events, Version 5.0 using the following categorical descriptors:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.
Grade 4: Life-threatening consequences; urgent intervention indicated.
Grade 5: Death related to adverse event.

### Pharmacokinetics

Plasma samples are used to evaluate the PK of lazertinib and serum samples are used to evaluate the PK of amivantamab. Samples collected for PK may additionally be used to evaluate safety or efficacy aspects that address concerns arising during or after the study period. Participant confidentiality will be maintained.

### Evaluations

### Lazertinib

Plasma samples are collected for the evaluation of PK of lazertinib at the designated timepoints according to the Schedule of Activities (Table 3). Potential metabolites, including M7, may be evaluated. Individual PK parameters are estimated, and descriptive statistics calculated for each dose level. Based on the individual plasma concentration-time data, using the actual sampling times, the following PK parameters of lazertinib are derived by noncompartmental analysis:
Cmax: maximum plasma concentration
Ctrough: plasma concentration immediately prior the next intervention administration.
tmax: time to reach the maximum plasma concentration.
AUC (0-t): area under the plasma concentration-time curve from time zero to time t
AUCτ: area under the plasma concentration-time curve during a dose interval time period (τ) at steady-state.
CLss/F: total apparent clearance of study intervention at steady-state.
Vss/F: apparent volume of distribution at steady-state.
R: accumulation ratio

Additional plasma PK parameters could be determined as appropriate. In addition, a population pharmacokinetic-based modeling approach may also be applied for PK analysis.

### Amivantamab

Serum samples are collected for the evaluation of PK and immunogenicity of amivantamab at the designated timepoints according to the Schedule of Activities (Table 3). Individual PK parameters are estimated, and descriptive statistics calculated. Based on the individual serum concentration-time data, using the actual sampling times, the following PK parameters of amivantamab are derived by noncompartmental analysis:
Cₘₐₓ: maximum serum concentration
Ctrough: serum concentration immediately prior the next intervention administration

Additional serum PK parameters could be determined as appropriate. In addition, a population pharmacokinetic-based modeling approach may also be applied for PK analysis.

Additional information about the collection, handling, and shipment of biological samples can be found in the laboratory manual.

### Analytical Procedures

Plasma and serum samples are analyzed to determine concentrations of lazertinib and amivantamab, respectively using a validated, specific, and sensitive (eg, liquid chromatography with tandem mass spectrometry [LC-MS/MS], liquid chromatography-mass spectrometry [LC-MS], Immunoassay) method by or under the supervision of the sponsor.

If required, some plasma and serum samples may be analyzed to document the presence of circulating metabolites using a qualified research method. In addition, plasma and serum PK samples may be stored for future analysis of other coadministered treatments and protein binding and the metabolite profile.

### Pharmacodynamics and Biomarkers

Screening blood samples from all participants undergoing ctDNA analysis to evaluate pretreatment mutational status of EGFR, cMet, and other key oncogenes to characterize the tumor. Additional blood samples are collected during the study and may be evaluated for ctDNA to assess changes in the levels or types of genetic alterations observed over time, and to monitor for the emergence of potential markers of resistance to the study therapy.

Blood samples are collected at timepoints specified in the Schedule of Activities, Error! Reference source not found. Changes in circulating markers may be assessed in pre and posttreatment samples and levels correlated with response to study interventions. PBMCs may be used to determine the correlation between amivantamab activity and polymorphisms in Fc-gamma receptor IIIa, a receptor expressed on certain immune cells that may interact with amivantamab.

Collected tumor tissue samples may be evaluated for biomarkers relevant to cancer and/or analyzed to confirm ctDNA results. Levels of EGFR and cMet protein may also be assessed in tissue samples.

### Immunogenicity Assessments

Serum samples are screened for antibodies binding to amivantamab and the titer of confirmed positive samples reported. Other analyses may be performed to further characterize the immunogenicity of amivantamab.

Antibodies to amivantamab are evaluated in serum samples collected from all participants according to the Schedule of Activities, Table 3. Additionally, serum samples should also be collected at the final visit from participants who discontinued study intervention or were withdrawn from the study.

Serum samples are used to evaluate the immunogenicity of amivantamab.

### Analytical Procedures

The detection and characterization of antibodies to amivantamab are performed using a validated assay method by or under the supervision of the sponsor. All samples collected for detection of antibodies to amivantamab are evaluated for amivantamab serum concentration to enable interpretation of the immunogenicity data. Antibodies may be further characterized and/or evaluated for their ability to neutralize the activity of the study intervention(s).

### 7. Statistical Considerations

A general description of the statistical methods to be used to analyze the efficacy and safety data are outlined below.

Data will be summarized using descriptive statistics. Continuous variables will be summarized using the number of observations, mean, SD, coefficient of variation, median, and range as appropriate. Categorical values will be summarized using the number of observations and percentages as appropriate.

### Statistical Hypotheses

The hypothesis in the Phase 1b LACP combination cohort is that lazertinib can be safely administered with amivantamab and standard of care chemotherapy, with the probability of DLT<33.3%, to participants with advanced or metastatic EGFR-mutated NSCLC who failed EGFR TKI.

### Sample Size Determination

In the Phase 1b LACP combination cohort, up to 6 participants are enrolled to achieve at least 3 DLT-evaluable participants at the first dose level of lazertinib (240 mg). Additional participants may be dosed in subsequent cohorts, with lazertinib initiated at reduced doses of 160 mg and then 80 mg, if needed. Once tolerability is demonstrated, up to a total of 20 participants may be enrolled to further characterize the safety, tolerability, and PK of lazertinib at the recommended dose in combination with amivantamab and chemotherapy (RP2ChD), following study review and agreement.

### Populations for Analyses

For purposes of analysis, the following analysis sets are defined:

The ‘full’ analysis set consists of participants who receive at least one dose of any study intervention. This population is considered as primary efficacy analysis and is used for all analyses unless otherwise specified.

The ‘DLT-evaluable’ analysis set consists of participants that receive any amount of study intervention during the first 21 days (Phase 1) from the first infusion of study intervention.

The ‘response-evaluable’ analysis set consists of all participants who receive at least one dose of study intervention and who have at least one postbaseline disease assessment, clinical progression, or died due to disease progression before the first postbaseline disease assessment.

The ‘PK’ analysis set consists of participants who receive at least one dose of study intervention and have at least 1 evaluable concentration measurement of lazertinib or amivantamab.
Participants will be removed from the estimation of certain PK parameters on an individual basis due to, for eg, missing PK samples such that the PK parameters cannot be appropriately derived. These participants will be identified at the time of the analyses along with their reason for removal.

The ‘immunogenicity’ analysis set consists of all participants who receive at least one dose of amivantamab and have at least one post-infusion immunogenicity sample.

### Statistical Analyses

### Primary Endpoints

The primary endpoint of the study, by study Phase, are:
Frequency of DLT
Adverse events defined by the NCI-CTCAE Criteria, Version 5.0 (LACP combination cohort only)

### Secondary Endpoints

The secondary endpoints of the study are:
Plasma lazertinib PK parameters
Serum amivantamab PK parameters.
Incidence of antibodies to amivantamab.

### Safety Analyses

All participants who were assigned to study intervention and received at least one dose of the study intervention will be included in the safety and tolerability analysis.

Baseline for all laboratory evaluations, vital signs, and ECG measurements will be defined as the last evaluation done before the first study intervention administration.

### Adverse Events

Any AE occurring at or after the initial administration of study intervention through the day of last dose plus 30 days is considered to be treatment-emergent (TE). All reported TEAEs will be included in the analysis. For each adverse event, the percentage of participants who experience at least 1 occurrence of the given event will be summarized by system organ class, preferred term, worst grade experienced by the participant.

Parameters with predefined NCI-CTCAE Version 5.0 toxicity grades will be summarized. Change from baseline to the worst adverse event grade experienced by the participant during the study will be provided as shift tables.

### Clinical Laboratory Tests

Laboratory data will be summarized by type of laboratory test. Descriptive statistics will be calculated for each laboratory analyte at baseline and for observed values and changes from baseline at each scheduled timepoint. Changes from baseline results will be presented in pre-versus postintervention cross-tabulations (with classes for below, within, and above normal ranges).

Frequency tabulations of the abnormalities will be made. A listing of participants with any laboratory results outside the reference ranges will be provided. A listing of participants with any markedly abnormal laboratory results will also be provided.

Parameters with predefined NCI-CTCAE, Version 5.0 toxicity grades will be summarized. Change from baseline to the worst adverse event grade experienced by a participant during the study will be provided as shift tables.

### Electrocardiogram

The effects on cardiovascular variables will be evaluated by means of descriptive statistics and frequency tabulations. These tables will include observed values and changes from baseline values at each scheduled timepoint (the predose ECG will be used as baseline) in ECG parameters, including heart rate, QRS axis, and intervals for PR interval, QT interval, QRS interval, RR interval, and QTcF. Frequency tabulations of the abnormalities will be made.

Descriptive statistics of QTc intervals and changes from baseline will be summarized at each scheduled timepoint. The percentage of participants with QTc interval >450 milliseconds, >480 milliseconds, or >500 milliseconds will be summarized, as will the percentage of participants with QTc interval increases from baseline >30 milliseconds or >60 milliseconds.

### Vital Signs

Descriptive statistics of temperature, pulse/heart rate, respiratory rate, SpO₂ (pulse oximetry) and blood pressure (systolic and diastolic) values and changes from baseline will be summarized at each scheduled timepoint. The percentage of participants with values beyond clinically important limits will be summarized.

### Efficacy Analyses

ORR is defined as the proportion of participants who achieve either a complete (CR) or partial response (PR) in the full analysis set (or response-evaluable analysis set for interim monitoring), as defined by investigator assessment using RECIST v1.1. Observed ORR along with their two-sided 95% exact confidence intervals will be presented. Confirmation of Investigator assessed ORR may be performed through IRC if indicated.

Clinical benefit rate (CBR) is defined as the percentage of participants achieving complete or partial response, or durable stable disease (duration of at least 11 weeks) as defined by RECIST v1.1. Observed ORR and CBR, along with their two-sided 95% confidence intervals, will be presented for each cohort and dose level as appropriate.

Time to event endpoints including PFS, duration of response (DOR), time to treatment failure (TTF), and overall survival (OS) will be estimated using the Kaplan-Meier method. DOR will be calculated as time from initial response of CR or PR to progressive disease (PD) or death due to any cause, whichever comes first, only for participants who achieve CR or PR. Progression free survival (PFS) is defined as the time from first infusion of study intervention to PD or death due to any cause. TTF is defined as the time from the first administration of the study intervention to discontinuation of treatment for any reason, including disease progression, treatment toxicity, death, and will be utilized to capture clinical benefit for patients continuing treatment beyond RECIST v1.1 defined disease progression. OS is defined as the time from first infusion of study intervention to death due to any cause. For time-to-endpoint endpoints, Kaplan-Meier estimates will be presented graphically, and median time to event, along with corresponding 95% CIs, will be obtained from the Kaplan-Meier estimates. Confirmation of Investigator assessed DOR, PFS, and CBR may be performed through IRC if indicated.

### Pharmacokinetic Analyses

### Pharmacokinetic Analytical Procedures (Lazertinib and Amivantamab)

All plasma and serum concentrations below the lowest quantifiable concentration or missing data will be labeled as such in the concentration data presentations or statistical analysis system dataset. Concentrations below the lowest quantifiable concentration will be treated as zero in the summary statistics.

For each compound, plasma or serum concentration data will be listed for participants and descriptive statistics will be summarized for each nominal timepoint by cohort in tables of mean, SD, median, and range over time, as appropriate. PK parameters will be estimated for participants, and descriptive statistics will be calculated by cohort.

Mean or median concentration versus time profiles will be plotted, and individual concentration-time profiles may also be plotted. Population PK modeling of plasma or serum concentration-time data may be performed using nonlinear mixed-effects modeling if appropriate. Data may be combined with those of other selected studies. The results of any population PK analyses will be presented in a separate report.

### Immunogenicity Analyses

The incidence of antibodies to amivantamab will be summarized for all participants who receive at least 1 dose of amivantamab and have appropriate samples for detection of antibodies to amivantamab (ie, participants with at least 1 sample obtained after their first dose of amivantamab).

A listing of participants who are positive for antibodies to amivantamab will be provided. The maximum titers of antibodies to amivantamab will be summarized for participants who are positive for antibodies to amivantamab.

Other immunogenicity analyses may be performed to further characterize the immune responses that are generated.

### Example 2. Amivantamab and Lazertinib in Combination with Platinum-Based Chemotherapy in Relapsed/Refractory EGFR-Mutant NSCLC

### Methods

The LACP (lazertinib, amivantamab, carboplatin, pemetrexed) cohort of the CHRYSALIS-2 study (Clinicaltrials_gov identifier NCT04077463) enrolled patients with EGFR-mutant NSCLC whose disease progressed on or after treatment with an EGFR TKI as last line of therapy (maximum of 3 prior lines). Patients received 1400 mg (1750 mg for bodyweight ≥80 kg) intravenous amivantamab weekly for the first 4 doses (first dose was split as 350 mg on day 1 and 1050 mg (1400 mg for body weight ≥80 kg) on day 2 of cycle 1) up to cycle 2 day 1 and then 1750 mg (2100 mg for bodyweight ≥80 kg) every 3 weeks (21-day cycle) thereafter, in combination with 240 mg oral lazertinib daily, and 500 mg/m2 pemetrexed with carboplatin (AUC5). Carboplatin treatment was stopped after 4 cycles. Adverse events were graded using CTCAE, v.5. Response in patients who had at least 1 postbaseline disease assessment will be assessed by the investigator per RECIST v1.1.

### Results

Enrolled patients have received a median of 2 (range, 1-3) prior lines of therapy, including osimertinib (n=14), gefitinib (n=3), and afatinib (n=3). To date, at a minimum follow-up of 3 months, best responses include 10 patients with confirmed partial response, 7 with stable disease, and 3 with progressive disease. The most common treatment-emergent adverse events were infusion related reaction (73.3%), neutropenia (66.7%), rash (46.7%), thrombocytopenia (40.0%), fatigue and nausea (33.3% each). Of the 5 participants discontinued from treatment, 2 were because of chemotherapy-related serious adverse events and 3 were because of progressed disease.

Amivantamab in combination with lazertinib and chemotherapy yielded high overall response rates in patients who progressed on EGFR TKIs as prior line of therapy. The safety profile of the LACP regimen was consistent with the individual agents, with no evidence of new safety signals or additive toxicity.

### Example 3. Amivantamab in Combination with Chemotherapy in Patients with Advanced NSCLC

Amivantamab administered on a 21-day cycle in combination with chemotherapeutic agents carboplatin and pemetrexed in subjects with advanced non-small cell lung cancer (NSCLC) amivantamab in combination with chemotherapy was investigated in CHRYSALIS study (Clinicaltrials_gov identifier NCT02609776).

### Methods

Patients had advanced NSCLC and were eligible for platinum-based chemotherapy in accordance with standard of care. amivantamab was dosed weekly at 1400 mg (1750 mg for body weight ≥80 kg) for the first 4 doses, then at 1750 mg (2100 mg for body weight ≥80 kg) every 3 weeks (Q3W) in combination with pemetrexed (500 mg/m2) and carboplatin (AUC 5; up to cycle 4) in a 21-day cycle. The first amivantamab dose was split as 350 mg on day 1 and 1050 mg (1400 mg for body weight ≥80 kg) on day 2 of cycle 1. Response was assessed by investigator per RECIST v1.1. Tolerability was assessed using a 3+3 dose de-escalation design.

### Results

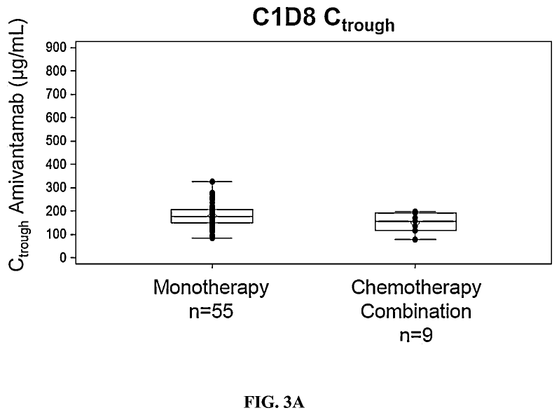

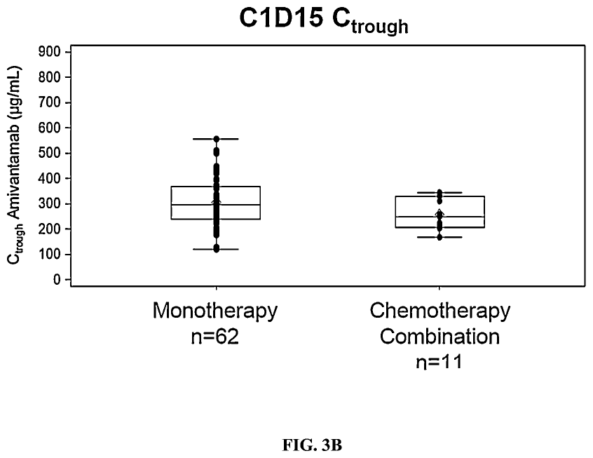

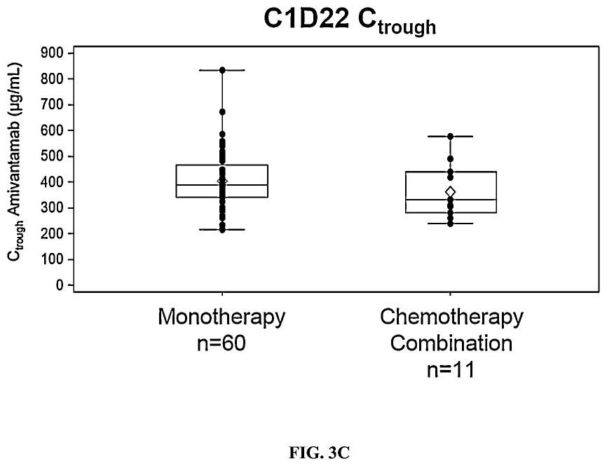

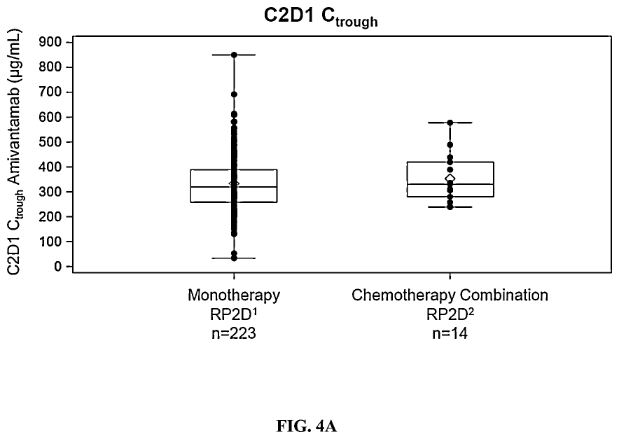

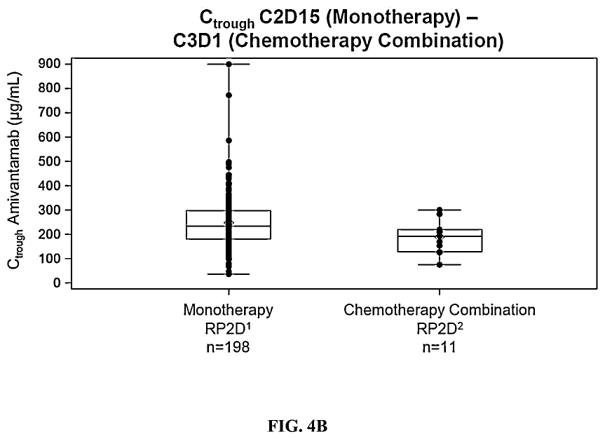

Of the 5 patients who were initially dosed, 3 were evaluable for dose-limiting toxicity (DLT); no DLTs were observed, and dose expansion up to 20 patients has been initiated. Twenty patients (5 with bodyweight ≥80 kg) had received the combination; 16 patients completed 4 cycles and went on to maintenance: 15 were beyond cycle 6 and 1 patient beyond cycle 13. Median age was 62 years (range: 36-83 y). Nineteen patients had EGFR mutations, including Exon 19 deletion (9 patients), L858R (2 patients), Exon 20 insertion (7 patients), and Exon 20 S768I (1 patient). One patient had a KRAS mutation (G13R). Four had history of brain metastases, and 15 had ≥1 prior lines of therapy (LOT), with 9 heavily-pretreated (2-7 prior LOT). Median duration of treatment was 5.6 months (range: 0.1-9.2 months). Most common treatment-emergent adverse events (TEAEs) were infusion-related reaction (65%) and rash (60% dermatitis acneiform+30% rash). Twelve patients (44%) had grade ≥3 TEAEs; most frequent events collectively reflected anticipated cytopenias (45% neutropenia, 6% anemia, 6% thrombocytopenia)—one patient discontinued carboplatin due to anemia. Preliminary cycle 1 pharmacokinetic (PK) data (n=9) suggest no impact of chemotherapy on amivantamab exposure (FIG. 3A-C). Preliminary trough concentration comparisons suggest that higher doses of amivantamab given Q3W (21-day cycle), are similar to the recommended dose for monotherapy given every 2 weeks (1050 (body weight <80 kg)/1400 mg (body weight >80 kg) QW in cycle 1 (C1) (28-day cycle), followed by 1050/1400 mg Q2W in C2 and beyond (FIG. 4A-B).

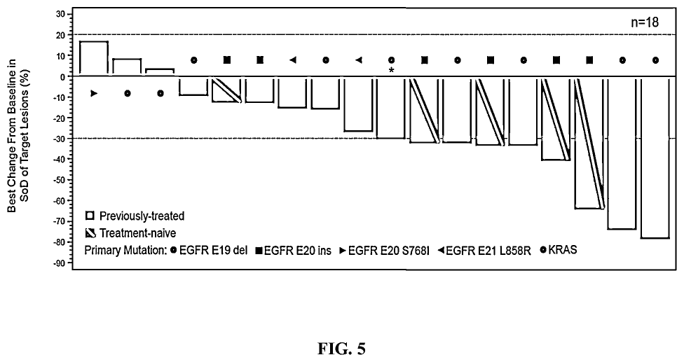

Eighteen patients were evaluable for response (FIG. 5). One patient discontinued before the first disease assessment and another patient had non-measurable disease; neither are included in the plot (FIG. 5). Antitumor activity was observed across heterogenous population. Among 18 response evaluable patients, 8 reported best response of PR (partial reasponse). Among 5 patients with treatment naïve, EGFR Exon20ins positive disease, 4 patients had best response of PR (FIG. 5).

In summary, no dose limiting toxicities were observed, and the RP2DQ3W for amivantamab was established (1750 mg for <80 kg and 2100 mg for ≥80 kg). amivantamab in combination with chemotherapy was tolerable, with a toxicity profile consistent with that observed with each therapy alone. Antitumor activity was observed in diverse NSCLC population, including in treatment naïve patients with EGFR Exon20ins NSCLC. amivantamab exposure was not impacted by chemotherapy. Similar amivantamab exposure was achieved with 28 day Q2W and 21 day Q3W dosing.

We claim:

1. A method of treating an epidermal growth factor receptor (EGFR)-expressing or hepatocyte growth factor receptor (c-Met)-expressing cancer in a subject in need thereof, comprising administering to the subject
an isolated bispecific anti-EGFR/c-Met antibody comprising a first domain comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and a second domain comprising a HCDR1 of SEQ ID NO: 7, a HCDR2 of SEQ ID NO: 8, a HCDR3 of SEQ ID NO: 9, a LCDR1 of SEQ ID NO: 10, a LCDR2 of SEQ ID NO: 11 and a LCDR3 of SEQ ID NO: 12, at a dose of about 1400-2100 mg, administered once per a 21-day cycle.

2. The method of claim 1, wherein the method further comprises administration of a 3^{rd} generation EGFR tyrosine kinase inhibitor (TKI).

3. The method of claim 2, wherein the 3^{rd} generation EGFR TKI is of formula (I):

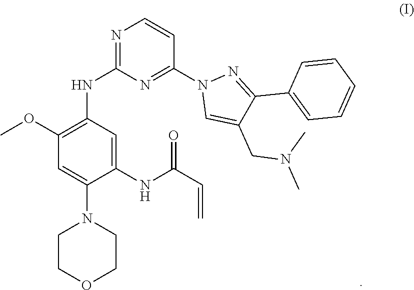 [SMILES: C=CC(=O)Nc1cc(Nc2nccc(-n3cc(CN(C)C)c(-c4ccccc4)n3)n2)c(OC)cc1N1CCOCC1]

4. The method of claim 3, wherein the 3^{rd} generation EGFR TKI is administered at a dose of 240 mg daily.

5. The method of claim 1, wherein the starting dose of the bispecific anti-EGFR/c-Met antibody is a split dose given over two consecutive days, wherein
on day one (D1) of the first cycle, the antibody is administered at a dose of about 350 mg, and on day two (D2) of the first cycle, the antibody is administered at a dose of about 1050 mg if the subject weighs less than 80 kg, or
on D1 of the first cycle, the antibody is administered at a dose of about 350 mg, and on D2 of the first cycle, the antibody is administered at a dose of about 1400 mg if the subject weighs 80 kg or more.

6. The method of claim 1, wherein the method further comprises an initial cycle (Cycle 1) comprising administering the bispecific antibody once a week for three weeks at a dose of about 1400 mg if the subject weighs less than 80 kg, or at a dose of about 1750 mg if the subject weighs 80 kg or more.

7. The method of claim 6, wherein the method further comprises a second cycle (Cycle 2) comprising administering the bispecific antibody once in three weeks at a dose of about 1400 mg if the subject weighs less than 80 kg, or at a dose of about 1750 mg if the subject weighs 80 kg or more.

8. The method of claim 1, wherein the method comprises administering the bispecific antibody once every 3 weeks at a dose of about 1750 mg if the subject weighs less than 80 kg, or at a dose of about 2100 mg if the subject weighs 80 kg or more.

9. The method of claim 1, wherein the method further comprises one or more chemotherapeutic agents comprising platinum.

10. The method of claim 9, wherein the one or more chemotherapeutic agents comprise pemetrexed and/or carboplatin.

11. The method of claim 9, wherein the one or more chemotherapeutic agents are pemetrexed and carboplatin, administered at day 1 of each 21-day cycle.

12. The method of claim 9, wherein the one or more chemotherapeutic agents are pemetrexed administered at a dose of about 500 mg/m² and carboplatin administered to achieve area under the curve (AUC) 5.

13. The method of claim 9, wherein the one or more chemotherapeutic agents are pemetrexed administered at a dose of about 500 mg/m² and carboplatin AUC 5, administered at day 1 of each cycle up to and including cycle four (Cycle 4), and wherein pemetrexed is administered at a dose of about 500 mg/m² at day 1 of each subsequent cycle.

14. The method of claim 1, wherein the first domain of the bispecific anti-EGFR/c-Met antibody binds EGFR and the second domain binds c-Met.

15. The method of claim 14, wherein the first domain that binds EGFR comprises a heavy chain variable region (VH) of SEQ ID NO: 13 and a light chain variable region (VL) of SEQ ID NO: 14 and the second domain that binds c-Met comprises the VH of SEQ ID NO: 15 and the VL of SEQ ID NO: 16.

16. The method of claim 15, wherein the bispecific anti-EGFR/c-Met antibody is an IgG1 isotype.

17. The method of claim 15, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

18. The method of claim 15, wherein the bispecific anti-EGFR/c-Met antibody has a biantennary glycan structure with a fucose content of between about 1% to about 15%.

19. The method of claim 3, wherein the compound of formula (I) or solvate, hydrate, tautomer, or a pharmaceutically acceptable salt thereof is lazertinib mesylate represented by a compound of formula (II)

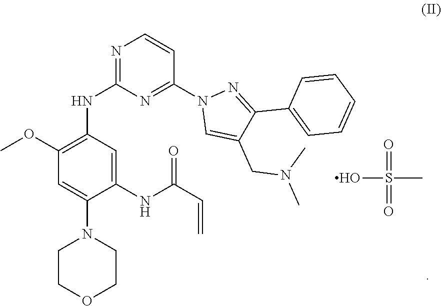

20. The method of claim 19, wherein the compound of formula (I) or solvate, hydrate, tautomer, or a pharmaceutically acceptable salt thereof is N-(5-(4-(4-((dimethylamino)methyl)-3-phenyl-1H-pyrazol-1-yl)pyrimidin-2-ylamino)-4-methoxy-2-morpholinophenyl) acrylamide.

21. The method of claim 1, wherein the EGFR or c-Met expressing cancer is a lung cancer.

22. The method of claim 21, wherein the cancer is a non-small cell lung cancer (NSCLC).
